SEQUENCE LISTING (60 sequences)

SEQ ID NO: 1 - DNA, 24 bp, Artificial Sequence
  Artificial Template T1
agagtccaccaagcttcagattag

SEQ ID NO: 2 - DNA, 24 bp, Artificial Sequence
  Artificial Template T2
agagtccaccaagctctagattag

SEQ ID NO: 3 - DNA, 15 bp, Artificial Sequence
  Probe A1
tctgaagctttgatc

SEQ ID NO: 4 - DNA, 18 bp, Artificial Sequence
  Probe A2
taatctgaagctttgatc

SEQ ID NO: 5 - DNA, 14 bp, Artificial Sequence
  Probe A3
ctagagctttgatc

SEQ ID NO: 6 - DNA, 13 bp, Artificial Sequence
  PROBE A4
tgaagctttgatc

SEQ ID NO: 7 - DNA, 12 bp, Artificial Sequence
  Probe A5
gaagctttgatc

SEQ ID NO: 8 - DNA, 27 bp, Artificial Sequence
  PROBE B1
aaaagatcaaattggtggactctaaaa

SEQ ID NO: 9 - DNA, 26 bp, Artificial Sequence
  PROBE B2
aaagatcaaattggtggactctaaaa

SEQ ID NO: 10 - DNA, 25 bp, Artificial Sequence
  PROBE B3
aagatcaaattggtggactctaaaa

SEQ ID NO: 11 - DNA, 24 bp, Artificial Sequence
  PROBE B4
agatcaaattggtggactctaaaa

SEQ ID NO: 12 - DNA, 23 bp, Artificial Sequence
  PROBE B5
gatcaaattggtggactctaaaa

SEQ ID NO: 13 - DNA, 20 bp, Artificial Sequence
  PROBE B6
aaaagatcaaattggtggac

SEQ ID NO: 14 - DNA, 27 bp, Artificial Sequence
  PROBE B7
aaaagatcaaattggtggactctaaaa

SEQ ID NO: 15 - DNA, 27 bp, Artificial Sequence
  PROBE B8
aaaagatcaaattggtggactctaaaa

SEQ ID NO: 16 - DNA, 27 bp, Artificial Sequence
  PROBE B9
aaaagatcaaattggtggactctaaaa

SEQ ID NO: 17 - DNA, 27 bp, Artificial Sequence
  PROBE B10
aaaagatcaaattggtggactctaaaa

SEQ ID NO: 18 - DNA, 27 bp, Artificial Sequence
  PROBE B11
gggggatcaaattggtggactctaaaa

SEQ ID NO: 19 - DNA, 27 bp, Artificial Sequence
  PROBE B12
ccccgatcaaattggtggactctaaaa

SEQ ID NO: 20 - DNA, 27 bp, Artificial Sequence
  PROBE B13
ttttgatcaaattggtggactctaaaa

SEQ ID NO: 21 - DNA, 43 bp, Artificial Sequence
  PROBE B14
agtaacggaaaaccgttactaaaagatcaaattggtggactct

SEQ ID NO: 22 - DNA, 41 bp, Artificial Sequence
  PROBE B15
agtaacggaaaaccgttactaagatcaaattggtggactct

SEQ ID NO: 23 - DNA, 39 bp, Artificial Sequence
  PROBE B16
tgtaacggaaaaccgttacagatcaaattggtggactct

SEQ ID NO: 24 - DNA, 43 bp, Artificial Sequence
  PROBE B17
agtaacggaaaaccgttactaaaagatcaatttggtggactct

SEQ ID NO: 25 - DNA, 43 bp, Artificial Sequence
  PROBE B18
agtaacggaaaaccgttactaaaagatcatattggtggactct

SEQ ID NO: 26 - DNA, 39 bp, Artificial Sequence
  PROBE B19
tgtaacggaaaaccgttacagatcaatttggtggactct

SEQ ID NO: 27 - DNA, 39 bp, Artificial Sequence
  PROBE B20
tgtaacggaaaaccgttacagatcatattggtggactct

SEQ ID NO: 28 - DNA, 39 bp, Artificial Sequence
  PROBE B21
tgtaacggaaaaccgttacagatcattttggtggactct

SEQ ID NO: 29 - DNA, 39 bp, Artificial Sequence
  PROBE B22
tggatcggaaaaccgatccagatcatattggtggactct

SEQ ID NO: 30 - DNA, 51 bp, Artificial Sequence
  PROBE B23
tggatcggcagatcgcaaaagctgccgatccagatcatattggtggactct

SEQ ID NO: 31 - DNA, 59 bp, Artificial Sequence
  PROBE B24
cagatcgcaaaagcgatctgtggatcggaaaaccgatccagatcatattggtggactct

SEQ ID NO: 32 - DNA, 79 bp, Artificial Sequence
  PROBE B25
ctgatccgaaaacggatcagcagatcgcaaaagcgatctgtggatcggaaaaccgatccagatcatattggtggactct

SEQ ID NO: 33 - DNA, 95 bp, Artificial Sequence
  PROBE B26
tggatcggctgatccgaaaacggatcagcagatcgcaaaagcgatctggagatcggaaaaccgatctcccgatccagatcatattggtggactct

SEQ ID NO: 34 - DNA, 24 bp, Artificial Sequence
  Artificial Template T2
agagtccaccaagctctagattag

SEQ ID NO: 35 - DNA, 39 bp, Artificial Sequence; identical to SEQ ID NO: 29
  Probe Strand B

SEQ ID NO: 36 - DNA, 14 bp, Artificial Sequence
  Probe Strand A
ctagagctttgatc

SEQ ID NO: 37 - DNA, 24 bp, Artificial Sequence
  Artificial Template T2
agagtccaccaagctctagattag

SEQ ID NO: 38 - DNA, 27 bp, Artificial Sequence
  Probe Strand B
aaaagatcaaattggtggactctaaaa

SEQ ID NO: 39 - DNA, 14 bp, Artificial Sequence
  Probe Strand A
ctagagctttgatc

SEQ ID NO: 40 - DNA, 20 bp, Artificial Sequence
  Molecular Beacon probe
tcaaatctgaagctttgatc

SEQ ID NO: 41 - DNA, 24 bp, Artificial Sequence
  Artificial Template T1
agagtccaccaagcttcagattag

SEQ ID NO: 42 - DNA, 20 bp, Artificial Sequence
  Molecular Beacon probe
tcaaatctgaagctttgatc

SEQ ID NO: 43 - DNA, 24 bp, Artificial Sequence
  Artificial Template T1
agagtccaccaagcttcagattag

SEQ ID NO: 44 - DNA, 39 bp, Artificial Sequence; identical to SEQ ID NO: 29
  Probe Strand B

SEQ ID NO: 45 - DNA, 15 bp, Artificial Sequence
  Probe Strand A
tctgaagctttgatc

SEQ ID NO: 46 - DNA, 24 bp, Artificial Sequence
  Artificial Template T1
agagtccaccaagcttcagattag

SEQ ID NO: 47 - DNA, 23 bp, Artificial Sequence
  Probe Strand B
gatcaaattggtggactctaaaa

SEQ ID NO: 48 - DNA, 23 bp, Artificial Sequence
  Probe Strand A
ctaatctgaagctttgauctttt

SEQ ID NO: 49 - DNA, 24 bp, Artificial Sequence
  Template
agagtccaccaagcttcagattag

SEQ ID NO: 50 - DNA, 23 bp, Artificial Sequence
  Probe Strand B
gatcaaattggtggactctaaaa

SEQ ID NO: 51 - DNA, 23 bp, Artificial Sequence
  Probe Strand A
ctaatctgaagctttgauctttt

SEQ ID NO: 52 - DNA, 24 bp, Artificial Sequence
  Artificial Template T1
agagtccaccaagcttcagattag

SEQ ID NO: 53 - DNA, 23 bp, Artificial Sequence
  Probe Strand B
gatcaaattggtggactctaaaa

SEQ ID NO: 54 - DNA, 23 bp, Artificial Sequence
  Probe Strand A
ctaatctgaagctttgauctttt

SEQ ID NO: 55 - DNA, 24 bp, Artificial Sequence
  Artificial Template T1
agagtccaccaagcttcagattag

SEQ ID NO: 56 - DNA, 23 bp, Artificial Sequence
  Probe Strand B
gatcaaattggtggactctaaaa

SEQ ID NO: 57 - DNA, 23 bp, Artificial Sequence
  Probe Strand A
ctaatctgaagctttgatctttt

SEQ ID NO: 58 - RNA, 26 nt, Artificial Sequence
  Artificial Micro RNA Template transcribed with T7 RNA polymerase from artificial DNA template containing a T7 promoter sequence
ggagaguccaccaagcuucagauuag

SEQ ID NO: 59 - DNA, 23 bp, Artificial Sequence
  Probe Strand B
gatcaaattggtggactctaaaa

SEQ ID NO: 60 - DNA, 19 bp, Artificial Sequence
  Probe Strand A
ctaatctgaagctttgatc

(19) United States
(12) Patent Application Publication
(10) Pub. No.: US 2011/0207132 A1
(43) Pub. Date: Aug. 25, 2011

(54) PROBES AND METHODS FOR DETECTING ANALYTES

(75) Inventor: Herman Sintim, Silver Spring, MD (US)

(73) Assignee: UNIVERSITY OF MARYLAND, College Park, MD (US)

(21) Appl. No.: 13/057,417

(22) PCT Filed: Aug. 4, 2009
(86) PCT No.: PCT/US09/52765

§ 371 Date: Feb. 3, 2011

Related U.S. Application Data
(60) Provisional application No. 61/086,040, filed on Aug. 4, 2008.

(51) Int. Cl. C12Q 1/68; C07H 21/00

(52) U.S. Cl. 435/6.11; 536/24.3

(57) ABSTRACT

Embodiments disclosed herein relate generally to probes, methods, and kits for detecting the presence of a target analyte. The probe generally comprises two strands that have regions of complementarity and do not associate with each other at the reaction temperature. In the presence of an analyte, the two strands of the probe can hybridize to each other, and the analyte can hybridize to both strands of the probe in a juxtapose manner to form a tripartite structure (probe-analyte complex). If the region of complementarity between the two probe strands contain cognate restriction endonuclease (REN) sequences, then the formation of the tripartite structure will lead to the generation of a REN site that can be cleaved by a REN, and the cleavage can then be detected by a variety of methods to signal the presence of the analyte.

### CROSS-REFERENCE TO RELATED APPLICATIONS

The present application claims priority to U.S. Provisional Application No. 61/086,040, filed Aug. 4, 2008, which is hereby incorporated by reference in its entirety.

### FIELD OF THE INVENTION

The present application relates generally to compositions and methods for detecting the presence of and quantitating nucleic acid analytes.

### BACKGROUND

The majority of commercial DNA detection methods require polymerase chain reaction (PCR) or ligase chain reaction (LCR) steps to preamplify the genes of interest, which are usually minor components in a complex mixture of other nucleic acid sequences. However, PCR or LCR based methods have limitations. For example, certain alleles amplify better than others under PCR conditions leading to over- or underestimation of the concentration of a target DNA sequence in a sample. In addition, the use of thermal cycling in both PCR and LCR limits the adaptation of these methods, for example, for in vivo applications.

The aforementioned limitations of PCR and LCR based detection methods have spurred the development of alternative detection platforms that either do not require preamplification of the gene of interest or achieve non-PCR isothermal amplified sensing. The majority of methods that do not pre-amplify the DNA target are based on the difference between the melting temperature of perfectly matched and mismatched DNA templates. This usually requires stringent temperature control, sophisticated instrumentation/reagents, or the need for a skilled technician. Therefore the adoption of these technologies has been limited.

### SUMMARY OF THE INVENTION

Embodiments herein relate to a probe for detecting a nucleic acid analyte in a sample referred to as a junction probe. The junction probe generally comprises two strands that have regions of complementarity and do not associate with each other at the reaction temperature. In the presence of an analyte, the two strands of the junction probe can hybridize to each other, and the analyte can hybridize to both strands of the junction probe in a juxtapose manner to form a tripartite structure (probe-analyte complex). If the region of complementarity between the two probe strands contain cognate restriction endonuclease (REN) sequences, then the formation of the tripartite structure will lead to the generation of a REN site that can be cleaved by a REN, and the cleavage can then be detected by a variety of methods.

In some embodiments, the probe comprises: (a) a first nucleic acid probe strand comprising a first analyte recognition region that is substantially complementary to a first portion of the nucleic acid analyte; and a first cleavage region comprising a first cleavage site sequence; and (b) a second nucleic acid probe strand comprising a second analyte recognition region that is substantially complementary to a second portion of the nucleic acid analyte; and a second cleavage region that is substantially complementary to the first cleavage region; wherein the probe is configured such that in the presence of the nucleic acid analyte, the first nucleic acid probe strand, the second nucleic acid probe strand and the nucleic acid analyte can hybridize to each other to form a probe-analyte complex, wherein: the first analyte recognition region is hybridized to the first portion of the nucleic acid analyte to form a first duplex region; the second analyte recognition region is hybridized to the second portion of the nucleic acid analyte to form a second duplex region; and the first cleavage region is hybridized to the second cleavage region to form a third duplex region, thereby forming a first cleavage site in the first cleavage region of the first nucleic acid probe strand; and wherein the melting temperature of the probe-analyte complex is greater than the melting temperature of a duplex of the first and second nucleic acid probe strands in the absence of the nucleic acid analyte.

In some embodiments, the probe (e.g., the first nucleic acid probe strand) further comprises a detectable moiety. In some embodiments, the detectable moiety comprises a fluorophore. In some embodiments, the first nucleic acid probe strand further comprises a second moiety, such as a quencher. In some embodiments, one of the moiety pair (e.g., fluorophore and quencher) are located upstream of the first cleavage site and the other is located downstream of the first cleavage site. In some embodiments, the detectable moiety comprises a biotin molecule. In some embodiments, the probe (e.g., the first nucleic acid probe strand) further comprises a solid support. In some embodiments, the detectable moiety comprises a macromolecular tag. For example, the macromolecular tag (e.g., horseradish peroxidase) can have enzymatic activity that can be readily detected or measured.

In some embodiments, the first cleavage site is located in the third duplex region. In some embodiments, the first cleavage site is located about 1 to about 6 nucleotides from the 5′ end or the 3′ end of the first cleavage region of the first nucleic acid probe strand. In some embodiments, the first cleavage site is located about 3 nucleotides from the 5′ end of the first cleavage region of the first nucleic acid probe strand.

In some embodiments, the detectable moiety is located about 0 to about 6 nucleotides from the 5′ end or the 3′ end of the first cleavage region of the first nucleic acid probe strand. In some embodiments, the detectable moiety is located at the 5′ end of the first cleavage region of the first nucleic acid probe strand. In some embodiments, the second nucleic acid probe strand further comprises a second cleavage site. In some embodiments, the second nucleic acid probe strand further comprises a phosphorothioate or a phosphorodithioate linkage at the second cleavage site.

In some embodiments, the second nucleic acid probe strand further comprises a first overhang region at the 5′ end of the second nucleic acid probe strand. In some embodiments, the first overhang region is single-stranded. In some embodiments, a portion of the first overhang region forms one or more fourth duplex regions. In some embodiments, the fourth duplex region comprises one or more nuclease resistant cleavage sites comprising the first cleavage site sequence. In some embodiments, the overhang region comprises a loop region adjacent to the duplex region. In some embodiments, the loop region comprises about 1 to about 10 nucleotides. In some embodiments, the loop region comprises about 4 nucleotides.

In some embodiments, the first duplex region comprises about 3 to about 30 nucleotides. In some embodiments, the first duplex region comprises about 7 nucleotides.

In some embodiments, the second duplex region comprises about 3 to about 30 nucleotides. In some embodiments, the second duplex region comprises about 15 nucleotides.

In some embodiments, the third duplex region comprises about 4 to about 30 nucleotides. In some embodiments, the third duplex region comprises about 7 nucleotides. In some embodiments, the third duplex region comprises a mismatched base pair. In some embodiments, at least one of the first or second duplex regions comprises a mismatched base pair.

In some embodiments, the first duplex region has a lower melting temperature than the second duplex region and wherein the third duplex region has a lower melting temperature than the second duplex region.

Additional embodiments relate to a kit comprising a probe described herein and instructions for use. In some embodiments, the kit further comprises an enzyme that cleaves a double stranded nucleic acid having the first cleavage site sequence.

Other embodiments relate to a method for detecting a nucleic acid analyte in a sample comprising: contacting the sample with a probe described herein; providing an enzyme that cleaves at the first cleavage site, wherein the first nucleic acid probe strand is cleaved into a first fragment and a second fragment; and detecting the cleavage of the first nucleic acid probe strand, thereby detecting the presence of the nucleic acid analyte.

The cleavage of the first nucleic acid probe strand, or a detectable moiety on the strand can be detected by any method known in the art. In some embodiments, the cleavage or moiety is detected by a method selected from the group consisting of fluorescent spectroscopy, light scattering spectroscopy, capillary electrophoresis, gel electrophoresis, colorimetry, mass spectrometry, gel electrophoresis, capillary electrophoresis, autoradiography, electrochemical detection (e.g., current, voltage, capacitance, or magnetoresistance measurement), surface plasmon resonance, surface enhanced Raman spectroscopy, piezoelectric sensing, chemiluminescence, bioluminescence, cantilever sensing, and dipstick. In some embodiments, the functionalities that are on the resulting cleavage products, such as 3′-OH and 5′-phosphate, can be utilized in a second amplification cycle that use, for example, ligases or polymerases.

In some embodiments, detection of the nucleic acid analyte is used to quantify the nucleic acid analyte.

In some embodiments, the enzyme comprises an endonuclease. In some embodiments, the endonuclease is a restriction endonuclease.

In some embodiments, the nucleic acid analyte is associated with a disease or disorder.

Some embodiments relate to a method of forming a structure comprising hybridized nucleic acids, the method comprising: providing a nucleic acid analyte comprising a first portion and a second portion; and providing a probe as described herein, wherein the probe is configured such that in the presence of the nucleic acid analyte, the first nucleic acid probe strand, the second nucleic acid probe strand and the nucleic acid analyte can hybridize to each other to form a probe-analyte complex, as described herein.

Some embodiments relate to a method for detecting a nucleic acid analyte in a sample, comprising contacting the sample with a probe, wherein the probe comprises: (a) a first nucleic acid probe strand comprising: a first fragment of an enzyme; a first probe recognition region and a first analyte recognition region that is substantially complementary to a first portion of the nucleic acid analyte; (b) a second nucleic acid probe strand comprising: a second fragment of the enzyme; a second probe recognition region that is substantially complementary to the first probe recognition region of the first nucleic acid probe strand; and a second analyte recognition region that is substantially complementary to a second portion of the nucleic acid analyte; wherein the probe is configured such that in the presence of the nucleic acid analyte, the first nucleic acid probe strand, the second nucleic acid probe strand and the nucleic acid analyte can hybridize to each other to form a probe-analyte complex, wherein: the first analyte recognition region of the first nucleic acid probe strand is hybridized to the first portion of the nucleic acid analyte to form a first duplex region; the second analyte recognition region of the second nucleic acid probe strand is hybridized to the second portion of the nucleic acid analyte to form a second duplex region; the first probe recognition region of the first nucleic acid probe strand is hybridized to the second probe recognition region of the second nucleic acid probe strand to form a third duplex region, thereby reconstituting the enzyme; and wherein the melting temperature of the probe-analyte complex is greater than the melting temperature of a duplex of the first and second nucleic acid probe strands in the absence of the nucleic acid analyte; and measuring the enzyme activity, thereby detecting the presence of the nucleic acid analyte.

In some embodiments, the enzyme comprises G-quadruplex DNAzyme.

### BRIEF DESCRIPTION OF THE DRAWINGS

FIG. 1 illustrates an embodiment of a detection method using a probe described herein.

FIG. 2 illustrates an embodiment of the detection method wherein the probe comprises a G-quadraplex DNAzyme.

FIG. 3 illustrates an embodiment of the detection method wherein the probe comprises protein peroxidase.

FIG. 4 illustrates an embodiment of the detection method wherein the probe comprises a G-quadraplex DNAzyme and protein peroxidase.

FIG. 5 shows analyte detection by a probe comprising two nucleic acid strands, probe A (A) and probe B (B). The probe demonstrated sequence selectivity using fluorescence based detection (FIG. 5A) and gel based detection (FIG. 5B). NT indicates no template (analyte) was added to the reaction.

FIG. 6A-C demonstrate the cleavage rate of probe-analyte complexes with different modifications at cleavage site B.

FIG. 7 demonstrates the cleavage rates of the embodiment described in FIG. 2 in the presence and absence of an analyte (template).

FIGS. 8A and 8B demonstrates the effect of the length of template hybridization region of probe A on cleavage rates.

FIG. 9 compares the detection of analytes by junction probes with the detection of analytes by molecular beacons.

FIG. 10A and FIG. 10B illustrates embodiments of the detection method wherein the probe comprises various restriction endonuclease cleavage sites.

FIG. 11A and FIG. 11B illustrate an embodiment of the detection method wherein the probe comprises an RNase H endonuclease cleavage site.

FIG. 12A and FIG. 12B illustrate an embodiment of the detection method wherein the probe comprises an RNase HII endonuclease cleavage site.

FIG. 13A and FIG. 13B illustrate an embodiment of the detection method wherein the analyte detected is microRNA.

### DETAILED DESCRIPTION OF VARIOUS EMBODIMENTS

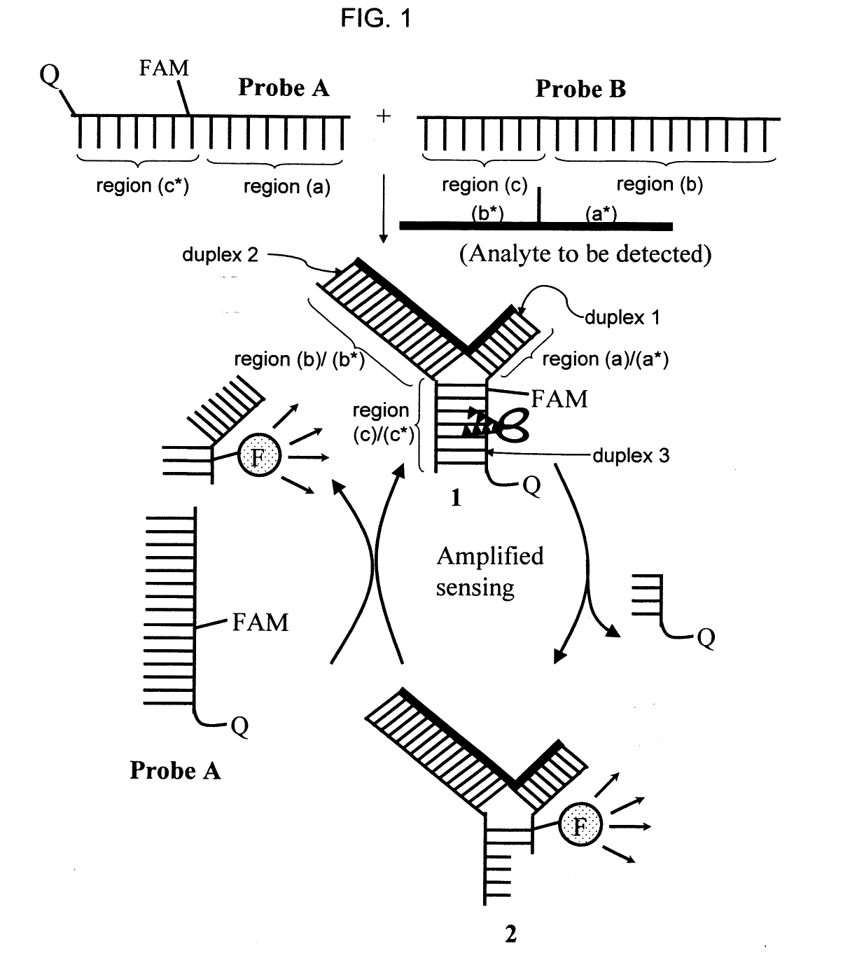

Embodiments disclosed herein relate to various probes and methods for detecting analytes using the probes described herein. In general, a probe comprises two strands (e.g., probe A and probe B) that have regions of complementarity and do not associate with each other at the reaction temperature and only hybridize to each other in the presence of an analyte that binds to both probes in a juxtapose manner. If the region of complementarity between the two probes A and B contain a restriction endonuclease (REN) sequence, then the formation of tripartite structure (probe-analyte complex (see, e.g., FIG. 1)), will lead to the generation of a cognate REN site that is cleaved by a REN, and the cleavage can then be detected by a variety of methods.

In some embodiments, the probes provide for the ability to detect analytes with a higher degree of specificity than conventional probes, thereby reducing erroneous background signal produced by the probe in the absence of the analyte. In some embodiments, enhanced specificity is provided by using probes comprising two or more nucleic acid strands. As a result, the probes described herein can be particularly useful in, for example, the detection of single nucleotide polymorphisms (SNPs).

In some embodiments, the probes and methods described herein provide amplification of the target signal. In some embodiments, probes can be used to amplify the signal obtained when detecting target analytes that are present in low quantities in cells or samples are detected. In various embodiments, the probes and methods described herein can detect picomolar concentrations (femtomoles) of nucleic acids. Any of the detection methods provided herein can be conducted without amplifying the analyte.

In some embodiments, the probes and methods described herein provide for isothermal detection. Any of the detection methods provided herein can be conducted under isothermal conditions. As a result, the probes described herein can be particularly useful in, for example, point-of care applications and in vivo detection methods where thermal cycling is not practical.

In some embodiments, methods for detecting analytes provide for the use of an enzyme (e.g., a restriction endonuclease) to cleave a probe-analyte complex that comprises a cleavage site located in the duplex region formed between the probe strands that can be cleaved by the enzyme. As a result, probes can be designed for analytes without consideration for the presence of a cleavage site sequence for the enzyme in the target. In some embodiments, a probe can be designed such that the probe comprises cleavage site sequences not present in the analyte to ensure the analyte is not affected by the activity of the enzyme or that the enzyme is not affected by the analyte alone.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference for the material referenced herein, and in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

### DEFINITIONS

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As utilized in accordance with the embodiments provided herein, the following terms, unless otherwise indicated, shall be understood to have the following meanings:

The term “nucleic acid” refers to natural nucleic acids, artificial nucleic acids, analogs thereof, or combinations thereof. Nucleic acids may also include analogs of DNA or RNA having modifications to either the bases or the backbone. For example, nucleic acid, as used herein, includes the use of peptide nucleic acids (PNA). The term “nucleic acids” also includes chimeric molecules. Nucleic acids include, but are not limited to, DNA, cDNA, genomic DNA, mitochondrial DNA, RNA, mRNA, miRNA, siRNA, piwi-interacting RNA, rRNA, tRNA, snRNA, and viral RNA.

As used herein, the terms “polynucleotide,” “oligonucleotide,” and “nucleic acid oligomers” are used interchangeably and refer to single-stranded and double-stranded polymers of nucleic acids, including, but not limited to, 2′-deoxyribonucleotides (nucleic acid) and ribonucleotides (RNA) linked by internucleotide phosphodiester bond linkages, e.g., 3′-5′ and 2′-5′, inverted linkages, e.g., 3′-3′ and 5′-5′, branched structures, or analog nucleic acids. Polynucleotides have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, e.g., 5-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine.

A “gene” (e.g., a marker gene) or “coding sequence” or a sequence, which “encodes” a particular protein, is a nucleic acid molecule which is transcribed (in the case of DNA) and translated (in the case of mRNA) into a polypeptide in vitro or in vivo when placed under the control of appropriate regulatory or control sequences. The boundaries of the gene are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

As used herein, the terms “complementary” or “complementarity” are used in reference to polynucleotides (e.g., a sequence of nucleotides) related by the base-pairing rules. For example, the sequence “5′-A-G-T-3′” is complementary to the sequence “3′-T-C-A-5′.” The nucleic acid sequences can comprise natural nucleotides (including their hydrogen bonding bases A, C, G, T, or U) and/or modified nucleotides or bases. Complementarity may be “partial,” in which less than all of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. As used herein, a hybridizing nucleic acid sequence is “substantially complementary” when it is at least 80%, more preferably at least 85%, 90%, 91%, 92%, 93%, 94%, 95%, 96%, 97%, 98%, 99% or 100%, identical and/or includes no more than one non-Watson-Crick base pairing interaction to a reference sequence in the hybridizing portion of the sequences. In any of the embodiments disclosed herein, two sequences which form a duplex region preferably are, or preferably are at least, 80%, 85%, 90%, 91%, 92%, 93%, 94%, 95%, 96%, 97%, 98%, 99% or 100%, complementary, or a range defined by any two of the preceding values. When two sequences or regions thereof which hybridize are not the same size, percent complementarity can be calculated based on the number of complementary base pairs divided by the total number of nucleotides in the smaller sequence or region. For example, an 18 base oligonucleotide having 16 bases complementary when aligned to a 1500 base sequence is 88.8% complementary.

The terms “hybridize,” “hybridization,” and their cognates are used herein to refer to the pairing of complementary nucleic acids or bases. Hybridization and the strength of hybridization (e.g., the strength of the association between the nucleic acids) is influenced by such factors as the degree of complementarity between the nucleic acids, stringency of the hybridization conditions involved, the melting temperature (Tm) of the formed hybrid, and the G:C ratio within the nucleic acids. A hybridizing sequence preferably is, or is at least, 75%, 80%, 85%, 90%, 91%, 92%, 93%, 94%, 95%, 96%, 97%, 98%, 99% or 100% complementary and is matched according to the base pairing rules. It may contain natural and/or modified nucleotides and bases.

A “cell” or “target cell” refers to a cell that contains or may contain an analyte for detection. Examples of target cells include, for example without limitation, cells that contain a nucleic acid signature for a disease, such as, for example, mutant mRNA or fusion mRNA entities. Other examples include, but are not limited to, cells that contain higher-than-background levels of mRNA, peptides, polypeptides, antibodies or fragments thereof, signal cascade molecules, viral particles, bacteria and parasitic organisms.

A “cleavage site sequence” refers to a nucleotide sequence that can be recognized by an enzyme (e.g., an endonuclease such as a restriction endonuclease (“REN” or “REase”)). In some embodiments, a cleavage site sequence (e.g., a cleavage site sequence located on a single-stranded nucleic acid strand) is not recognized by an enzyme until the cleavage site sequence (e.g., a first cleavage site sequence) is located on a duplex region comprising a cognate cleavage site sequence (e.g., a second cleavage site sequence). Upon formation of the duplex region comprising the cognate cleavage site sequences, one or more “cleavage sites” can be formed that are recognized and cleaved by an enzyme (e.g., an endonuclease such as a REN). In some embodiments, a cleavage site can be modified to make the site nuclease resistant. Examples of such modifications include, for example, introducing a phosphoromonothioate or phosphorodithioate linkage at the cleavage site. Any method known in the art can be used to make a cleavage site nuclease resistant. Enzymes suitable for embodiments disclosed herein include, for example, endonucleases such as restriction endonucleases and RNAseH. Any appropriate endonuclease known in the art can be used in the embodiments described herein (see, for example, E. S. Vanamee et al. Handbook of Metalloproteins (2006), which is herein incorporated by reference in its entirety). Preferred endonucleases are those for which the cleavage and recognition sites are the same.

### Probes

Embodiments disclosed herein relate to compositions and methods that include a “probe” comprising two nucleic acid strands (e.g., a first nucleic acid probe strand and a second nucleic acid probe strand). In some embodiments, the nucleic acid strands are single-stranded. In some embodiments, the nucleic acid strands can form secondary structures such as duplex regions and loop regions. In some embodiments, the first nucleic strand contains a first cleavage region comprising a first cleavage site sequence. In some embodiments, the first nucleic strand contains a first analyte recognition region that is substantially complementary to a first portion of a nucleic acid analyte. In some embodiments, the second nucleic acid probe strand contains a second cleavage region that is substantially complementary to the first cleavage region of the first nucleic acid probe strand and a second analyte recognition region that is substantially complementary to a second portion of the nucleic acid analyte.

In some embodiments, the probe is configured such that in the presence of the nucleic acid analyte, the first nucleic acid probe strand, the second nucleic acid probe strand and the nucleic acid analyte can hybridize to each other to form a probe-analyte complex such that the first analyte recognition region of the first nucleic acid probe strand is hybridized to the first portion of the nucleic acid analyte to form a first duplex region; the second analyte recognition region of the second nucleic acid probe strand is hybridized to the second portion of said nucleic acid analyte to form a second duplex region; and the first cleavage region of the first nucleic acid probe strand is hybridized to the second cleavage region of the second nucleic acid probe strand to form a third duplex region, thereby forming a duplex comprising a first cleavage site in the first cleavage region of the first nucleic acid probe strand. In some embodiments, the first cleavage site is located in the third duplex region. In some embodiments, the first cleavage site is located about 1 to about 6 nucleotides from the 5′ end or the 3′ end of the first cleavage region of the first nucleic acid probe strand. In some embodiments, the first cleavage site is located about 3 nucleotides from the 5′ end of the first cleavage region of the first nucleic acid probe strand. The optimal position of a cleavage site on a nucleic acid strand for a particular probe and cleavage enzyme can be routinely determined by methods described in the art based on the disclosure and methods described herein.

A duplex region (e.g., the first, second, third, or fourth duplex region) refers to a region of complementarity between two nucleic acid strands such that the two nucleic acid strands can hybridize over the length of the duplex region. In some embodiments, the length of the duplex region can be from about 2 to about 1000 bases, preferably from about 2 to about 500 bases, more preferably from about 4 to about 100 bases, or more preferably from about 5 to about 50 bases. In some embodiments, the length of the duplex region can be any number of bases between about 2 and about 1000 bases. In some embodiments, the length of the duplex region can be, or can be at least, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases, or a range defined by any two of these values. The length of various duplex regions present in the probe-analyte complex can be the same, or one or more of the duplex regions can be a different size.

In some embodiments, the first duplex region comprises about 3 to about 30 nucleotides. In some embodiments, the first duplex region comprises about 7 nucleotides. In some embodiments, the second duplex region comprises about 3 to about 30 nucleotides. In some embodiments, the second duplex region comprises about 15 nucleotides. In some embodiments, the third duplex region comprises about 4 to about 30 nucleotides. In some embodiments, the third duplex region comprises about 7 nucleotides.

In some embodiments, complementarity between strands forming a duplex region need not be perfect. For example, stable duplexes may contain one or more mismatched base pairs and/or one or more unmatched bases. In any of the embodiments disclosed herein, the number of mismatches for any of the duplex regions can be, or can be at least, or is preferably not more than, 1, 2, 3, 4, 5, 6, 7, 8, 9, 10, or more, or a range defined by any two of these values.

In general, the reaction temperature is chosen for methods described herein such that the probe-analyte complex is relatively stable compared to the probe in the absence of the analyte and compared to the probe-analyte complex after the probe-analyte complex has been cleaved by an enzyme (e.g., a restriction endonuclease).

The reaction temperature for a particular assay can be optimized by one of skill in the art, in view of the disclosures herein, for example, by varying the length of the probe strands, the length of the duplex regions (e.g., between the probe strands or between a probe strand an the analyte), base composition and sequence of the probe strands, ionic strength, and incidence of mismatched base pairs in the duplex regions. Those skilled in the art of nucleic acid technology can determine duplex stability empirically considering a number of variables including, for example, the length of the oligonucleotide, base composition and sequence of the oligonucleotide, ionic strength, and incidence of mismatched base pairs. The optimum length of a duplex region can be designed by one of ordinary skill in the art, for example, to determine the desired assay conditions (e.g., strand displacement conditions or melting temperatures) or the desired signal to noise ratio. In the preferred embodiments, the melting temperature of said probe-analyte complex is greater than the melting temperature of a duplex of the first and second nucleic acid probe strands in the absence of the nucleic acid analyte, and the melting temperature of the probe-analyte complex after cleavage of the probe. The melting temperature for a particular assay can be optimized by one of skill in the art, in view of the disclosures herein, for example, by varying the length of the oligonucleotide, base composition and sequence of the oligonucleotide, ionic strength, and incidence of mismatched base pairs. For example, high stringency conditions are known in the art (see, for example, Maniatis et al., Molecular Cloning: A Laboratory Manual, 2d Edition, 1989, and Short Protocols in Molecular Biology, ed. Ausubel, et al., both of which are hereby incorporated by reference in their entireties). Stringent conditions are typically sequence-dependent and can be different in different circumstances. Longer sequences typically hybridize specifically at higher temperatures. An extensive guide to the hybridization of nucleic acids is found, for example, in Tijssen, Techniques in Biochemistry and Molecular Biology-Hybridization with Nucleic Acid Probes, “Overview of principles of hybridization and the strategy of nucleic acid assays” (1993). Typically, stringent conditions can be selected to be about 5-10° C. lower than the thermal melting point (Tₘ) for the specific sequence at a defined ionic strength pH. The Tₘ is the temperature (under defined ionic strength, pH and nucleic acid concentration) at which 50% of the nucleic acid strands complementary to each other hybridize to each other at equilibrium. Stringent conditions are typically those in which the salt concentration is less than about 1.0 M sodium ion concentration, typically about 0.01 to 1.0 M sodium ion concentration (or other salts) at pH of about 7.0 to about 8.3 and the temperature is at least about 30° C. for short complementary sequences (e.g. 10 to 50 nucleotides) and at least about 60° C. for long complementary sequences (e.g. greater than 50 nucleotides). Stringent conditions can also be achieved with the addition of destabilizing agents such as formamide. In other embodiments, less stringent hybridization conditions can be used. For example, moderate or low stringency conditions may be used, as are known in the art (see, for example Maniatis and Ausubel, supra, and Tijssen, supra). Any two complementary or substantially complementary sequences described herein can be hybridized according to the conditions described above, and appropriate modifications can be made by routine experimentation in view of the disclosure herein to achieve the desired hybridization conditions.

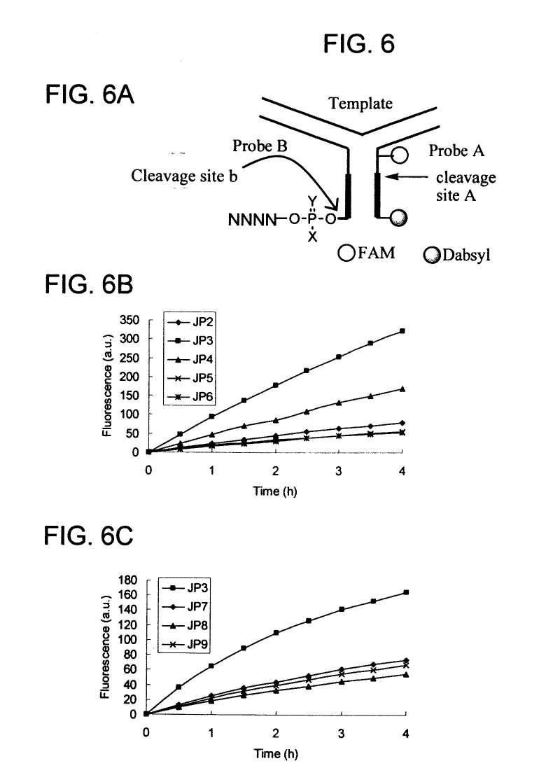

In some embodiments, one nucleic acid strand of the probe can extend beyond the other nucleic acid strand of the probe or the analyte, forming one or more overhang regions adjacent to a duplex region. In some embodiments the overhang region is located at the 5′ end of a nucleic acid strand (e.g., a strand of the probe or the analyte). In other embodiments, the overhang region is located at the 3′ end of a nucleic acid strand. For example, the second nucleic acid probe strand can comprises a first overhang region at the 5′-end of the second nucleic acid probe strand (see, for example, FIG. 6). In some embodiments, the overhang region is single-stranded, and in some embodiments all or a portion of the overhang is double-stranded. In some embodiments, a portion of the first overhang region forms 1, 2, 3, 4, or more duplex regions with itself (e.g., a fourth duplex region). In some embodiments, the duplex region(s) contain one or more cleavage sites or nuclease resistant cleavage sites. These cleavage sites can comprise the first cleavage site sequence of the first cleavage region of the first nucleic acid probe strand. In some embodiments, the overhang region comprises a loop region adjacent to the duplex region. In some embodiments, the loop region comprises, or comprises at least about 1, 2, 3, 4, 5, 6, 7, 8, 9, 10, 11, 12, 13, 14, 15, 16, 17, 18, 19, 20 or more nucleotides or a range defined by any two of the preceding values. In some embodiments, the loop region comprises about 4 nucleotides. In some embodiments, the length of the overhang region can be from about 1 to about 1000 bases, preferably from about 5 to about 500 bases, or more preferably from about 6 to about 100 bases, or more preferably from about 8 to about 80 bases. In some embodiments, the length of the overhang region can be any number of bases between about 2 and about 1000 bases. In some embodiments, the length of the overhang region can be, or can be at least, or can be less than, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases or a range defined by any two of the preceding values. The optimal length of the overhang region can be designed by one of ordinary skill in the art by methods described in the art and methods described herein, for example, to determine the desired assay conditions (e.g., strand displacement conditions) or the desired signal to noise ratio for a particular probe.

As is known in the art, a nucleoside is a base-sugar combination. The base portion of the nucleoside is normally a heterocyclic base sometimes referred to as a “nucleobase” or simply a “base.” The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally desired. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

A vast variety of modified nucleic acid analogs can also be used, including backbone modifications, sugar modifications, nitrogenous base modifications, or combinations thereof. In some embodiments, the portion of one or both of the probe strands that is complementary to the analyte is modified to increase affinity for the analyte, e.g., by including 2′-MOE sugar modifications.

### Modified Internucleoside Linkages (Backbones)

Specific examples of compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Suitable modified oligonucleotide backbones containing a phosphorus atom therein include, for example, phosphorothioates, chiral phosphorothioates, phosphorodithioates, phosphotriesters, aminoalkylphosphotriaminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Suitable modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Modified Sugar and Internucleoside Linkages-Mimetics

In other compounds, e.g., oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Further teaching of PNA compounds can be found in Nielsen et al., Science, 1991, 254, 1497-1500.

Further embodiments of the invention are oligonucleotides with phosphorothioate backbones and oligonucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester backbone is represented as —O—P—O—CH₂—). Also suitable are oligonucleotides having morpholino backbone structures.

### Modified Sugars

Modified compounds may also contain one or more substituted sugar moieties. The compounds of the invention can comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Also suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Another modification includes 2′-O-methoxyethyl (2′-O—CH₂CH₂OCH₃, also known as 2′-O-(2-methoxyethyl) or 2′-methoxyethoxy or 2′-MOE) (Martin et al., Helv. Chim. Acta, 1995, 78, 486-504) i.e., an alkoxyalkoxy group. Another modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A suitable 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotide compounds may also have sugar mimetics such as cyclobutyl moieties in place of the pentofuranosyl sugar.

A further modification of the sugar includes Locked Nucleic Acids (LNAs) in which the 2′-hydroxyl group is linked to the 3′ or 4′ carbon atom of the sugar ring, thereby forming a bicyclic sugar moiety. The linkage can be a methylene (—CH₂—)ₙ group bridging the 2′ oxygen atom and the 4′ carbon atom wherein n is 1 or 2.

### Natural and Modified Nucleobases

The compounds may also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-b)(1,4)benzoxazin-2(3H)-one), carbazole cytidine (2H-pyrimido(4,5-b)indol-2-one), pyridoindole cytidine (H-pyrido(3′,2′:4,5)pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Some of these nucleobases are particularly useful for increasing the binding affinity of the compounds of the invention. These include 5-substituted pyrimidines, 6-azapyrimidines and N2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently suitable base substitutions, even more particularly when combined with 2′-O-methoxyethyl sugar modifications.

### Conjugates

Another modification of the compounds of the invention involves chemically linking to the compound one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups of the invention include inter-calators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include, bu are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include, but are not limited to, groups that improve uptake, enhance resistance to degradation, and/or strengthen sequence-specific hybridization with the target nucleic acid. Groups that enhance the pharmacokinetic properties, in the context of this invention, include, but are not limited to, groups that improve uptake, distribution, metabolism or excretion of the compounds of the present invention. Conjugate moieties include, but are not limited to, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

Oligonucleotides used in the compositions of the present invention can also be modified to have one or more stabilizing groups that are generally attached to one or both termini of the oligonucleotides to enhance properties such as for example nuclease stability. Included in stabilizing groups are cap structures. By “cap structure or terminal cap moiety” is meant chemical modifications, which have been incorporated at either terminus of oligonucleotides. These terminal modifications protect the oligonucleotides having terminal nucleic acid molecules from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Suitable 3′-cap structures of the present invention include, for example, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925).

### Analytes

Some embodiments relate to methods for detecting an analyte or a plurality of analytes. The terms “analyte,” “target,” “target analyte,” and “detection target” referred to herein can be used interchangeably. In some embodiments, methods can be used to analyze biological analytes. In some embodiments, methods can be used to analyze non-biological analytes. Suitable biological analytes include, but are not limited to, nucleic acids, proteins, polypeptides, peptides, peptide nucleic acids, antibodies, antigens, receptors, molecules (e.g., a signal cascade molecule), hormones, biological cells, microorganisms (e.g., bacteria), parasitic organisms, cellular organelles, cell membrane fragments, bacteriophage, bacteriophage fragments, whole viruses, viral particles, viral fragments, and small molecules such as lipids, carbohydrates, amino acids, drug substances, and molecules for biological screening and testing. An analyte can also refer to a fused entity or a complex of two or more molecules, for example, a ribosome with both RNA and protein elements or an enzyme with substrate attached.

In preferred embodiments, the analyte is a nucleic acid molecule, such as DNA, cDNA, genomic DNA, mitochondrial DNA, RNA, mRNA, miRNA, siRNA, piwi-interacting RNA, rRNA, tRNA, snRNA, viral RNA, and fragments and segments thereof. An analyte or target sequence can be single-stranded, double-stranded, continuous, or fragmented, so long as a probe can be used to detect the target or a subsequence of the target. The target may be a gene, a gene fragment, or an extra-chromosomal nucleic acid sequence, for example. As used herein, a “subsequence” refers to a portion of a sequence, such as a target nucleic acid sequence, that is contained within the longer sequence.

In some embodiments, the analyte can be at least a portion of the sequence of any gene for which detection is desirable. In some embodiments, the detection target can be an mRNA. In other embodiments, the analyte can be DNA. In some embodiments, the analyte can be a portion of a nucleic acid (e.g., an mRNA) associated with a disease or disorder. In some embodiments, the analyte can be a portion of a nucleic acid not associated with a disease or disorder. In some embodiments, the analyte can be a non-biological analyte. Non-biological analytes include, but are not limited to, organic compositions, inorganic compositions and other compositions not typically found in a biological system.

Samples to be tested for analytes or sources of analytes (such as tissues or cells) can be from organisms and pathogens such as viruses and bacteria or from an individual or individuals, including, but not limited to, skin, blood, plasma, serum, spinal fluid, lymph fluid, synovial fluid, urine, tears, blood cells, organs, tumors, and also samples of in vitro cell culture constituents, such as conditioned medium resulting from the growth of cells in cell culture medium, recombinant cells and cell components. The presence of analytes can also be tested from environmental samples such as air or water samples, or from forensic samples from biological or non-biological samples, including clothing, tools, publications, letters, furniture, etc. Additionally, the presence of analytes can also be tested from synthetic sources. The probes and methods provided herein can be used in a variety of applications, such as commercial applications. For example, probes and methods described herein can be used at one or more steps in a production process to test either for one or more contaminants and/or to test for one or more desired components. The analytes can be provided in a sample that can be a crude sample, a partially purified or substantially purified sample, or a treated sample, where the sample can contain, for example, other natural components of biological samples, such as proteins, lipids, salts, nucleic acids, and carbohydrates. The presence of analytes can be tested, for example, in vitro, in situ, or ex vivo. In some embodiments, the presence of an analyte can be tested in vivo in a subject.

In some embodiments, the target analyte is preferably a nucleic acid molecule (e.g., an mRNA). In some embodiments, the nucleic acid analyte comprises a portion that is complementary or substantially complementary to a region of the first nucleic acid probe strand and a portion that is complementary or substantially complementary to a region of the second nucleic acid probe strand, such that in the presence of the analyte, the first nucleic acid probe strand, the second nucleic acid probe strand and the nucleic acid analyte can hybridize to each other to form a probe-analyte complex as described herein. In a preferred embodiment, the two portions of the analyte complementary or substantially complementary to the probe strands are adjacent, or separated by a number of nucleotides that is, or is at least, or is not more than, 1, 2, 3, 4, 5, 6, 7, 8, 9, 10, 11, 12, 13, 14, 15, 16, 17, 18, 19, 20, or more nucleotides, or a range defined by any two of the proceeding values. In some embodiments, the two complementary or substantially complimentary portions of the analyte are separated by a portion of the analyte which forms one or more secondary structures, such as a hairpin loop, which effectively brings the two portions in close proximity, allowing for the analyte-probe complex described herein to form.

In some embodiments, the analyte-binding region of the probe strand is preferably at least 80%, more preferably at least 85%, 90%, 91%, 92%, 93%, 94%, 95%, 96%, 97%, 98%, 99% or 100%, complementary to at least a portion of the analyte. In preferred embodiments, the analyte binding region is, or is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases in length, or a range defined by any two of the proceeding values.

The phrase “specifically bind(s)” or “bind(s) specifically” when referring to a detection probe refers to a detection probe that has intermediate or high binding affinity, exclusively or predominately, to a target molecule. The phrase “specifically binds to” refers to a binding reaction which is determinative of the presence of a target in the presence of a heterogeneous population of other biologics. Thus, under designated assay conditions, the specified binding region binds preferentially to a particular target and does not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically, a specific or selective reaction will be at least twice the background signal or noise and more typically more than 10 times the background signal or noise. Specific portions of the target sequence can be determined, for example, by BLAST (Basic Local Alignment Search Tool).

### Detectable Moieties

In some embodiments, one or both probe strands can contain one or more detectable moieties. As used herein, the term “moiety” refers to one or more molecules that can be attached to a probe strand (e.g., attached to a nucleotide) and can typically be detected. Examples of moieties include, but are not limited to, “labels,” and “signal altering moieties.” In certain embodiments, a label can be a moiety that produces a signal or that interacts with another moiety to produce a signal. In certain embodiments, a label can interact with another moiety to modify a signal of the other moiety. In certain embodiments, a label can bind to another moiety or complex that produces a signal or that interacts with another moiety to produce a signal. In certain embodiments, the first nucleic acid probe strand contains a label (detectable moiety), and the label emits a detectable signal or can be measured when the first nucleic acid probe strand of the probe-analyte complex is cleaved into at least two fragments. In certain embodiments, the label emits a detectable signal or can be measured upon strand displacement of the fragment (e.g., a first fragment of the first nucleic acid probe strand) comprising the detectable moiety.

In some embodiments, the probes contain one or more pairs of moieties. A “pair of moieties” or an “interactive label pair” refers to a pair of labels wherein at least one label exhibits a measurable characteristic upon activation of the probe (e.g., by binding of the probe to an analyte followed by a cleavage event as described herein). For example, the first nucleic acid probe strand can contain a first moiety (e.g., a detectable moiety such as a fluorophore) attached to a first nucleotide and a second moiety (e.g., a quencher) attached to a second nucleotide. In the absence of the analyte, the pair of moieties are in closer proximity for effective interaction, including, but not limited to, interaction between a molecular energy transfer pair or enzyme-inhibitor pair. In the presence of the analyte, the probe-analyte complex is formed, forming the first cleavage site on the first nucleic probe strand that can be cleaved by an enzyme (e.g., an endonuclease such as a restriction endonuclease). Cleavage of the first nucleic acid probe, for example, into a first fragment comprising a fluorophore and a second fragment comprising a quencher leads to a detectable change in signal. The signal can be measured, for example, when one or both of the first fragment comprising the fluorophore and the second fragment comprising the quencher are displaced from the probe-analyte complex. The detectable change in signal from a moiety pair can be used to detect or quantitate the target analyte. It will be understood that the labels can consist of multiple signal altering moieties if so desired.

A variety of signal altering moieties are suitable for use in the probe. For example, signal altering moieties can include a wide range of energy donor and acceptor molecules to construct resonance energy transfer probes. Energy transfer can occur, for example, through fluorescence resonance energy transfer, bioluminescence energy transfer, or direct energy transfer. Fluorescence resonance energy transfer occurs when part of the energy of an excited donor is transferred to an acceptor fluorophore which re-emits light at another wavelength or, alternatively, to a quencher group that typically emits the energy as heat. There is a great deal of practical guidance available in the literature for selecting appropriate donor-acceptor pairs for particular probes, as exemplified by the following references: Pesce et al., Eds., Fluorescence Spectroscopy (Marcel Dekker, New York, 1971); White et al., Fluorescence Analysis: A Practical Approach (Marcel Dekker, New York, 1970); and the like. The literature also includes references providing exhaustive lists of fluorescent and chromogenic molecules and their relevant optical properties, for choosing reporter-quencher pairs (see, for example, Berlman, Handbook of Fluorescence Spectra of Aromatic Molecules, 2nd Edition (Academic Press, New York, 1971); Griffiths, Colour and Constitution of Organic Molecules (Academic Press, New York, 1976); Bishop, Ed., Indicators (Pergamon Press, Oxford, 1972); Haugland, Handbook of Fluorescent Probes and Research Chemicals (Molecular Probes, Eugene, 1992) Pringsheim, Fluorescence and Phosphorescence (Interscience Publishers, New York, 1949); and the like. Further, there is extensive guidance in the literature for derivatizing acceptor and quencher molecules for covalent attachment via readily available reactive groups that can be added to a molecule. Many donor and acceptor molecules, in addition to synthesis techniques, are also readily available commercially.

In certain embodiments, the first signal altering moiety is a fluorophore and the second signal altering moiety is a fluorescence quencher. In the absence of a target analyte, the two signal altering moieties are close enough in space for effective molecular energy transfer and the fluorescent signal of the fluorophore is substantially suppressed by the fluorescence quencher. In the presence of a target analyte, following cleavage and dissociation, the two signal altering moieties are far enough apart from each other in space that fluorescent signal of the fluorophore is restored for detection.

In alternative embodiments, the first signal altering moiety and the second signal altering moieties are both fluorophores that emit a certain wavelength when in close proximity and a different wavelength when further apart.

Suitable fluorophores include, but are not limited to, Alexa Fluor dyes (Invitrogen), coumarin, fluorescein (e.g., 5-carboxyfluorescein (5-FAM), 6-carboxyfluorescein (6-FAM), 2′,4′,1,4,-tetrachlorofluorescein (TET), 2′,4′,5′,7′,1,4-hexachlorofluorescein (HEX), and 2′,7′-dimethoxy-4′,5′-dichloro-6-carboxyfluorescein (JOE)), Lucifer yellow, rhodamine (e.g., tetramethyl-6-carboxyrhodamine (TAMRA), and tetrapropano-6-carboxyrhodamine (ROX)), 4,4-difluoro-5,7-dimethyl-4-bora-3a,4a-diaza-s-indacene (BODIPY), DABSYL, DABCYL, cyanine (e.g., Cy3, Cy5, and Cy7), eosine, Texas red, ROX, quantum dots, anthraquinone, nitrothiazole, and nitroimidazole compounds, Quasar and Cal-fluor dyes, and dansyl derivatives. Combination fluorophores such as fluorescein-rhodamine dimmers are also suitable (Lee et al. (1997) Nucleic Acids Res. 25:2816). Exemplary fluorophores of interest are further described in WO 01/42505 and WO 01/86001. Fluorophores can be chosen to absorb and emit in the visible spectrum or outside the visible spectrum, such as in the ultraviolet or infrared ranges.

A fluorescence quencher is a moiety that, when placed very close to an excited fluorophore, causes there to be little or no fluorescence. Suitable quenchers described in the art include, but are not limited to, BLACK HOLE QUENCHERS™ (Biosearch Technologies), Iowa Black RQ, Iowa Black FQ, rhodamine, tetramethyl rhodamine, pyrene butyrate, eosine nitrotyrosine, ethidium, fluorescein, Malachite green, Texas Red, and DABCYL and variants thereof, such as DABSYL, DABMI and Methyl Red. Fluorophores can also be used as quenchers, because they tend to quench fluorescence when touching certain other fluorophores. Suitable quenchers can be, for example, either chromophores such as DABCYL or malachite green, or fluorophores that do not fluoresce in the detection range when the detection oligonucleotide segment is in the open conformation. Gold nanoparticles, for example, are also suitable as fluorescent quenchers.

Alternatively, labels may provide antigenic determinants, radioactive isotopes, non-radioactive isotopes, nucleic acids available for hybridization, altered fluorescence-polarization or altered light-scattering. Still other labels include those that are chromogenic, chemiluminescent or electrochemically detectable. In the absence of a target analyte, these labels are preferably unavailable for detection or can be detected at a different level than in the presence of the analyte.

In some embodiments, the detectable moiety comprises a macromolecular tag. For example, the macromolecular tag (e.g., horseradish peroxidase) can have enzymatic activity that can be readily detected or measured. Other tags include, for example, functional nucleic acid enzymes such as peroxidase-mimicking DNAzymes or affinity protein tags such as streptavidin or tags made up of polymers that are used for filtration purposes.

Methods available to label a probe will be readily apparent to those skilled in the art in view of the disclosures herein. In some embodiments, a nucleic acid base can be labeled with a moiety. For example, a first nucleic acid base can be labeled with a first moiety (e.g., a detectable moiety such as a fluorophore). In some embodiments, the first moiety (e.g., the detectable moiety) is located about 0 to about 6 nucleotides from the 5′ end or the 3′ end of the first cleavage region of the first nucleic acid probe strand. In some embodiments, the detectable moiety is located at the 5′ end or the 3′ end of the first cleavage region of the first nucleic acid probe strand. The optimal position of a moiety (e.g., a fluorophore) on a nucleic acid strand for a particular probe can be routinely determined by methods described in the art in view of the methods described herein.

In some embodiments, a second nucleic acid base can be labeled with a second moiety. In some embodiments, the first and second moieties can comprise a moiety pair (e.g., a fluorophore and a quencher). In some embodiments, the first and second nucleic acid bases are hybridized to each other, for example, in the duplex region of the probe. In some embodiments, the first and second nucleic acid bases containing the moiety pair are adjacent to each other, or are, are at least, or are not more than, 1, 2, 3, 4, 5, 6, 7, 8, 9, 10, 11, 12, 13, 14, 15, or more bases from each other or a range defined by any two of the preceeding values. In some embodiments, the first moiety (e.g., a fluorophore) is located upstream of said first cleavage site and the second moiety (e.g., a quencher) is located downstream of said first cleavage site; in other embodiments, their locations are reversed. In various embodiments, a moiety can comprise multiple molecules. For example, a moiety can comprise multiple quenchers.

### Methods for Detecting Analytes

Some embodiments disclosed herein relate to methods for detecting analytes using probes described herein. In general, a probe comprises two strands (e.g., probe A and probe B) that have regions of complementarity and do not associate with each other at the reaction temperature and only hybridize to each other in the presence of an analyte that binds to both probes in a juxtapose manner. If the region of complementarity between the two probes A and B contain a restriction endonuclease (REN) sequence, then the formation of tripartite structure (probe-analyte complex (see, e.g., FIG. 1)), will lead to the generation of a cognate REN site that is cleaved by a REN (FIG. 1 scissors), and the cleavage can then be detected by a variety of methods.

In some embodiments, a probe is utilized as illustrated in FIG. 1. In the depicted embodiment, the probe comprises two nucleic acid strands, probe A and probe B. Probe A contains two regions, regions (a) and (c*). Region (a) is complementary to region (a*) of the analyte, and region (c*) is complementary to region (c) of Probe B. In the embodiment shown in FIG. 1, probe A comprises a fluorescent molecule (FAM) that is quenched by Dabsyl (Q). The FAM and Dabsyl molecules are separated by a 7-mer sequence (5′-TTTGATC-3′: region (c*)) that contains a BfuCI restriction endonuclease (REN) cleavage sequence. Probe B is unlabeled and also contains two regions; a 7-mer region (region (c)) that is complementary to part of probe A (c*) and region (b) that is complementary to region (b*) of the analyte. BfuCI only cleaves double-stranded DNA; therefore, probe A cannot be cleaved by BfuCI unless it hybridizes with a complimentary strand to form a duplex. The melting temperature of the duplex that results from probe A annealing to probe B is lower than 15° C. Therefore, in the absence of a template that can enhance the hybridization of probe A and B, BfuCI will be unable to cleave probes A and B because the two oligonucleotides will predominately exist as single-stranded at the reaction temperatures (30° C.). However, in the presence of a DNA template comprising the analyte sequence, probes A and B and the template will hybridize to form a probe-analyte complex (ternary “Y” junction structure), complex 1. One arm of the probe-analyte complex contains a cleavage site for BfuCI and will be subsequently cleaved by this enzyme. The cleavage will then result in another ternary structure 2 which has a lower stability than complex 1. This results in the displacement of the fragment of probe A comprising the quencher from probe B, resulting in a detectable change in fluorescent signal because the FAM and Q are no longer in close proximity, and the FAM is no longer quenched. In some embodiments, upon cleavage of Probe A, the strands of structure 2 will displace, allowing the analyte to be used in further rounds of detection. This results in amplification of the signal.

In some embodiments, a first signal can be detected in the absence of the analyte and a second signal can be detected when the strands of the probe have been displaced after cleavage. In some embodiments, the presence of an analyte can be detected if the second signal is different (e.g., greater than or less than) than the first signal.

In some embodiments, the FAM and Q are replaced with other detectable moieties. For example, a radio-labeled fragment of probe A can be detected by gel electrophoresis and autoradiography. In some embodiments, upon cleavage of probe A, the strands of the second ternary structure will displace, allowing the analyte to be used in further rounds of detection. This can result in amplification of the signal. In an alternative embodiment, probe A can comprise an additional moiety, such as biotin, that remains on fragment 2 after the cleavage of probe A. The reaction mixture can be passed through a column that contains avidin-based beads. Radio-labeled uncleaved probe A will be retained by the column whereas cleaved probe A will pass through the column. The cleavage radio-labeled product can therefore be detected after a simple filtration process. In another embodiment, a macromolecular tag, or a solid support, is attached to probe A instead of using biotin labeling. After cleavage, the reaction mixture is simply filtered using a membrane with a cut-off that is lower than the macromolecular tag. Therefore, only the cleaved product (fragment 1) will pass through the membrane and can then be detected.

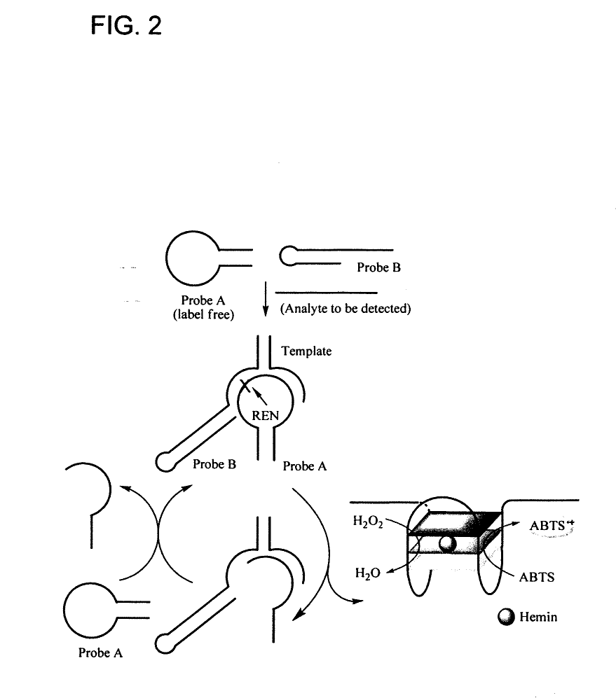

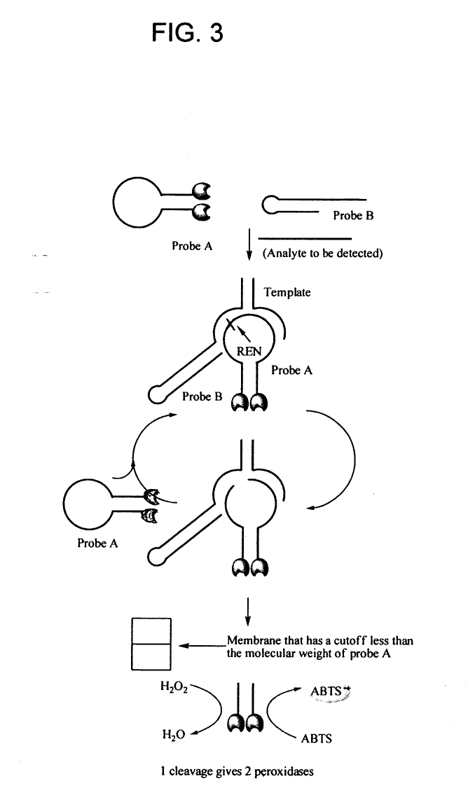

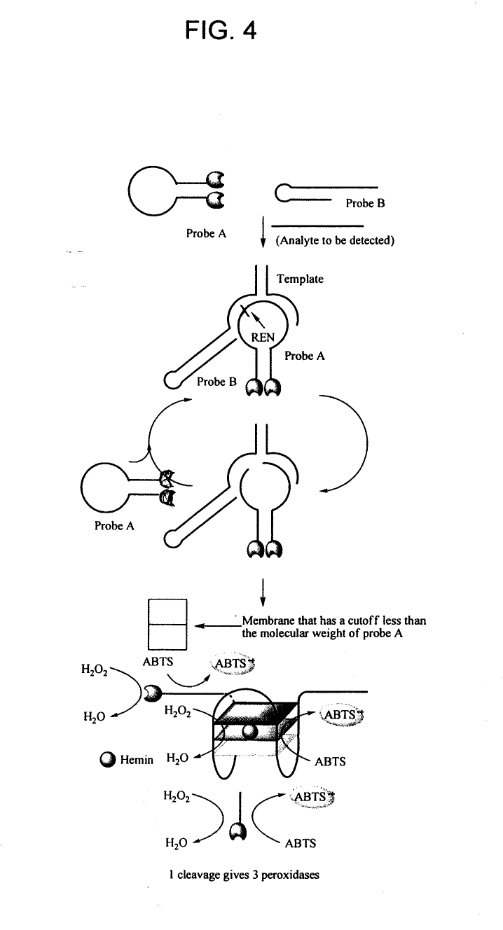

In some embodiments, a probe is utilized as illustrated in FIG. 2. In the depicted embodiment, the probe comprises two nucleic acid strands, probe A and probe B as described above, except that probe A is a locked G-quadruplex DNAzyme (the G-quadruplex DNAzyme cannot form because the alternative stem-loop structure is more stable). When the analyte is present, a ternary structure is formed. The ternary structure reconstitutes a restriction endonuclease cleavage site on the duplex between probe A and probe B and the subsequent cleavage by the REN leads to the destruction of the stem-loop probe A into two halves that fall off the ternary structure. Once probe A is cleaved into two halves, a G-quadruplex DNAzyme can readily form. This G-quadruplex DNAzyme can then act as a catalyst that oxidizes chromogenic substrates such as ABTS or fluorogenic substrates such as dihydrofluorescein. The use of G-quadruplex DNAzyme can be used to detect the cleavage product without using labeled probes. Additionally, G-quadruplex DNAzyme catalysis also provides a second layer of signal amplification. In alternative embodiments (FIG. 3), probe A can be labeled at both the 3′- and 5′-ends with a protein peroxidase that has a specific molecular weight, M. Therefore the total weight of probe A is greater than 2M+weight of DNA portion of probe A. After cleavage of the probe-analyte complex, the cleavage reaction can be filtered over a membrane with a cut off of M+(weight of DNA portion of probe A+1000). It follows that only the cleaved product can pass through the membrane. Once the cleavage product passes through the membrane, it can be detected via a peroxidation reaction. In further embodiments, the approaches described in FIG. 2 and FIG. 3 can be combined as illustrated in FIG. 4. After cleavage of the probe-analyte complex, three peroxidases (two protein-based and one nucleic acid-based (G-quadruplex DNAzyme)) can be produced that can pass through a membrane with a defined molecular weight cut-off. These peroxidases can then be detected via peroxidation reactions.

In some embodiments, a probe is utilized that comprises two nucleic acid strands, probe A and probe B as described above, except that probe A comprises a solid support and a restriction endonuclease (REN) cleavage sequence. As used herein, “solid support” means a surface to which a molecule can be irreversibly bound, including but not limited to membranes, sepharose beads, magnetic beads, silica based matrices, membrane based matrices, beads comprising surfaces including but not limited to styrene, latex or silica based materials and other polymers for example cellulose acetate, teflon, polyvinylidene difluoride, nylon, nitrocellulose, polyester, carbonate, polysulphone, metals, zeolites, paper, alumina, glass, polypropyle, polyvinyl chloride, polyvinylidene chloride, polytetrafluorethylene, polyethylene, polyamides, plastic, filter paper, dextran, germanium, silicon, (poly)tetrafluorethylene, gallium arsenide, gallium phosphide, silicon oxide, silicon nitrate and combinations thereof. Methods of attaching a solid support as defined herein are well known in the art. In the presence of a DNA template, probes A and B and the template will hybridize to form a probe-analyte complex (ternary “Y” junction structure). Cleavage results in the displacement of the fragment of probe A comprising the solid support (fragment 1) from probe B. Fragment 1 of probe A can then be detected by various means. For example, probe A can be immobilized on a surface comprising an electrode. In the presence of the analyte, the probe-analyte complex will be immobilized on the surface comprising the electrode. Upon cleavage of probe A by the enzyme, a change in electrochemical signal can be detected (e.g., a reduction in current, voltage, etc.). Alternatively, fragment 2 can be detected. For example, the reaction mixture can be washed such that uncleaved probe A and fragment 1 for probe A are retained on the solid support, while a labeled fragment 2 is collected in the wash and detected.

In some embodiments, the method comprises providing an enzyme that cleaves at a cleavage site on a probe strand. For example, a restriction endonuclease can cleave a first cleavage site present on the first nucleic acid probe strand of the probe-analyte complex. This results in the first nucleic acid probe strand being cleaved into a first fragment and a second fragment. Because the number of base-pairing is reduced, the resulting structure is not as stable as the probe-analyte complex and the resulting structure dissociates into binary and single components. In some embodiments, the components can be used to detect further analytes, thereby resulting in amplification of the signal.

Some embodiments provide for detecting multiple analytes in a sample. For example, various probes can be designed to test for various analytes in a sample, such that cleavage of each probe-analyte complex generates a unique fragment that can be measured by a variety of methods. For example, each fragment may be of a different size that can be detected, for example, by capillary electrophoresis. In some embodiments, each probe can be labeled with a unique detectable moiety (e.g., various fluorophores or combinations of fluorophores), such that cleavage of each probe-analyte complex generates a unique fluorescent signal that can be measured.

In some embodiments, the method comprises a probe that does not possess a detectable label. The presence of the analyte and cleavage of the probe-analyte complex can be detected, for example, by detecting one or more resulting fragments by gel electrophoresis and ethidium bromide staining.

In some embodiments, the methods for detecting analytes described herein can be used on a microarray. For example, binding of the analyte to the probe can displaces one or more moieties (e.g., quencher moiety and fluorescent moiety) to generate a fluorescent signal at that location on the chip.

Based on the disclosure herein, detection of cleavage or signals and changes in signals can be carried out by any method known in the art. For example, the detectable moiety can be detected by fluorescent spectroscopy, light scattering spectroscopy, capillary electrophoresis, gel electrophoresis, colorimetry, mass spectrometry, autoradiography, electrochemical detection (e.g., current, voltage, capacitance, or magnetoresistance measurement), surface plasmon resonance, surface enhanced Raman spectroscopy, piezoelectric sensing, chemiluminescence, bioluminescence, cantilever sensing, and dipstick. In some embodiments, the functionalities that are on the resulting cleavage products, such as 3′-OH and 5′-phosphate, can be utilized in a second amplification cycle that use, for example, ligases or polymerases.

### Delivery of Probes, Monomers, and Accessory Molecules to Target Cells

Probes, monomers, and any accessory molecules, such as, for example, helper molecules, can be formulated with any of a variety of carriers well known in the art to facilitate introduction into a sample (e.g., cells). Suitable carriers for delivery of nucleic acids to cells are well known in the art and include, for example, polymers, proteins, carbohydrates and lipids. For example, a cyclodextrin-containing polymer can be used for the delivery of the probes, monomers, and/or any accessory molecules. Commercial transfection reagents known in the art, such as, for example, LNCaP (Altogen Biosystems) or lipofectamine (Invitrogen), can be used.

Delivery of nucleic acids can be accomplished, for example, as described by Heidel (Heidel, J. D. 2005. Targeted, systematic non-viral delivery of small interfering RNA in vivo. Doctoral thesis, California Institute of Technology. 128 p., herein incorporated by reference in its entirety). Also contemplated within the scope of the subject matter are gene delivery systems as described by Felgner et al. (Felgner et al. 1997. Hum Gene Ther 8:511-512, herein incorporated by reference in its entirety), including cationic lipid-based delivery systems (lipoplex), polycation-based delivery systems (polyplex) and a combination thereof (lipopolyplex). Cationic lipids are described, for example, in U.S. Pat. Nos. 4,897,355 and 5,459,127, each of the foregoing which is herein incorporated by reference in its entirety. Proteins can also be used for nucleic acid delivery, such as synthetic neoglycoproteins (Ferkol et al. 1993. FASEB J 7:1081-1091; Perales et al. 1994. Proc Nat Acad Sci 91:4086-4090; each of which is incorporated herein by reference in its entirety), epidermal growth factor (EGF) (Myers, EPO 0273085, incorporated herein by reference in its entirety), and other ligands for receptor-mediated gene transfer (Wu and Wu. 1987. J Biol Chem 262(10):4429-4432; Wagner et al. 1990. Proc Natl Acad Sci USA 87(9):3410-3414; Ferkol et al. 1993. J. Clin Invest 92(5):2394-2300; Perales et al. 1994. Proc Natl Acad Sci USA 91(9):4086-4090; Myers, EPO 0273085; each of which is incorporated herein by reference in its entirety).

Viral and viral vector-like delivery systems generally known in the art, such as those described, for example, in U.S. Pat. No. 7,0333,834; U.S. Pat. No. 6,899,871; U.S. Pat. No. 6,555,367; U.S. Pat. No. 6,485,965; U.S. Pat. No. 5,928,913; U.S. patent application Ser. No. 10/801,648; U.S. patent application Ser. No. 10/319,074, and U.S. patent application Ser. No. 09/839,698, each of which is herein incorporated by reference, are also contemplated for use in the present subject matter. In addition, standard electroporation techniques can be readily adopted to deliver probes, monomers, and/or any accessory molecules.

Delivery of probes, monomers, and/or any accessory molecules can occur in vitro, in vivo or ex vivo. In some embodiments, cells can be removed from a patient and transfected with the probes, monomers, and/or any accessory molecules. In other embodiments, probes, monomers, and/or any accessory molecules can be delivered to cells in vivo such as by, for example, injection of the probes, monomers, and/or any accessory molecules within a delivery vehicle into the bloodstream or by intramuscular, subcutaneous, or intraperitoneal means. An appropriate means of delivering probes, monomers, and/or any accessory molecules to a desired population of cells can be identified by the skilled practitioner based on the particular circumstances without undue experimentation.

### Diagnostic Applications

Embodiments disclosed herein relate to diagnostic and prognostic methods for the detection of a disease or disorder and/or monitoring the progression of a disease or disorder. As used herein, the phrase “diagnostic” refers identifying the presence of or nature of a disease or disorder. The detection of an analyte (e.g., an mRNA) associated with a disease or disorder provides a means of diagnosing the disease or disorder. Such detection methods may be used, for example, for early diagnosis of the condition, to determine whether a subject is predisposed to a disease or disorder, to monitor the progress of the disease or disorder or the progress of treatment protocols, to assess the severity of the disease or disorder, to forecast the outcome of a disease or disorder and/or prospects of recovery, or to aid in the determination of a suitable treatment for a subject. The detection can occur in vitro or in vivo. In some embodiments, the probes and methods described herein can detect single nucleotide polymorphisms (SNPs). In some embodiments, the probes and methods described herein can detect genes or genetic mutations that are associated with a disease or condition.

Diseases contemplated for diagnosis in embodiments described herein include any disease in which an analyte, such as an analyte associated with the disease, is present in a cell, tissue, fluid or other sample described herein and can initiate formation of a probe-analyte complex. Preferred embodiments include, but are not limited to, diseases in which the analyte is a nucleic acid molecule. In some embodiments, the nucleic acid molecule is an mRNA molecule associated with a disease or disorder, such as a mutant mRNA molecule or foreign nucleic acid, for example bacterial or viral nucleic acids. However, disease-associated analytes can be, for example and without limitation, nucleic acid sequences, proteins, peptides, lipids, carbohydrates and small molecules.

In some embodiments, the disease to be diagnosed is a type of cancer, such as, for example, leukemia, carcinoma, lymphoma, astrocytoma, sarcoma and particularly Ewing's sarcoma, glioma, retinoblastoma, melanoma, Wilm's tumor, bladder cancer, breast cancer, colon cancer, hepatocellular cancer, pancreatic cancer, prostate cancer, lung cancer, liver cancer, stomach cancer, cervical cancer, testicular cancer, renal cell cancer, and brain cancer.

In other embodiments, the disease to be diagnosed is associated with infection by an intracellular parasite. For example, the intracellular parasite may be a virus such as, for example, an adenovirus, cytomegalovirus, Epstein-Barr virus, herpes simplex virus, human herpesvirus 6, varicella-zoster virus, hepatitis viruses, papilloma virus, parvovirus, polyomavirus, measles virus, rubella virus, human immunodeficiency virus (HIV), or human T cell leukemia virus. In other embodiments, the intracellular parasite may be a bacterium, protozoan, fungus, or a prion. More particularly, the intracellular parasite can be, for example, Chlamydia, Listeria, Salmonella, Legionella, Brucella, Coxiella, Rickettsia, Mycobacterium, Leishmania, Trypanasoma, Toxoplasma, and Plasmodium.

In some embodiments, a probe may be activated, either in vitro or in vivo, in response to the presence of an analyte. Thus, methods disclosed herein can be used for detecting the presence of an analyte in a sample or cells. In some embodiments, the methods can be used in forensics, environmental, and commercial applications.

### Compositions and Kits for Analyte Detection and Diagnosis

Compositions and kits for detecting the presence of an analyte are contemplated for use within the scope of the subject matter. In some embodiments, the compositions and/or kits comprise a probe and instructions for use. In some embodiments, the compositions and/or kits comprise a probe, one or more enzymes (e.g., an endonuclease such as a restriction endonuclease) and instructions for use. Upon delivery to a target cell or sample and recognition of the analyte by the probe, strand displacement of the probe is initiated by cleavage of the probe at least one cleavage site causing a change in signal detection that can readily be measured. In some embodiments, the kits can comprise one or more accessory molecules, such as a detectable moiety. In some embodiments, the probe can be configured for a particular analyte.

The compositions and/or kits can also contain other components, such as, for example, accessory molecules that facilitate analyte recognition and aid the triggering cleavage of a cleavage site or strand displacement of one or more strands of the probe. The compositions and/or kits may further comprise a reaction container, various buffers, and one or more reference samples (e.g., a negative and/or positive control).

Furthermore, the composition and/or kit can comprise a carrier that facilitates the introduction of nucleic acids, such as, for example, probes, enzymes and/or accessory nucleic acid molecules, into a cell, such as a cell containing an analyte associated with a disease or disorder. Carriers for delivery of nucleic acids and proteins into cells are well known in the art and examples are described above. In some embodiments, the kit is used to deliver probes, enzymes and/or accessory nucleic acid molecules to the tissues of a patient, wherein the tissues comprise cells comprising an analyte associated with a disease or disorder. In other embodiments, the kit is used to select for cells containing an analyte in vitro.

In some embodiments, the kit comprises reagents, containers, and/or instructions for preparing a sample for detection. For example, the kit can comprise the reagents for isolating nucleic acids from a biological sample such as cells, tissue, or fluids.

The following examples are offered for illustrative purposes only, and are not intended to limit the scope of the present invention in any way. Indeed, various modifications of the invention in addition to those shown and described herein will become apparent to those skilled in the art from the description herein and fall within the scope of the appended claims.

### Example 1

### Template Detection by Junction Probes is Sequence Selective

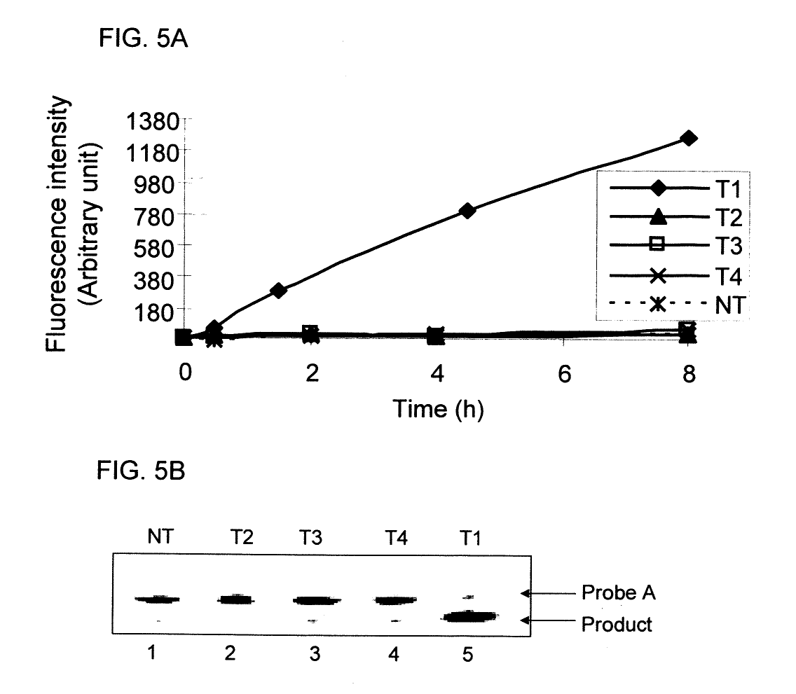

A probe comprising two nucleic acid strands, probe A and probe B, was synthesized and tested for its ability to recognize various targets described below in Table 1. The rate of cleavage at the REN site in the duplex region formed between probe A and B in the presence of 1 equiv of target sequence, T1, was 60 times higher than that in the absence of T1 (FIG. 5a). Template detection by junction probes was also sequence selective. When single mismatch targets (T2-T4, mismatch site bolded in Table 1) were used, the yields of cleavage products were similar to that of a target-free reaction, and only a matched template provided a significant product band on a PAGE gel (FIG. 5b). Similarly, the fluorescence intensities obtained with single mismatch targets were similar to that of target-free reaction (FIG. 5a).

TABLE 1
DNA template sequences

| | |
|---|---|
| T1 | 5′-AGAGTCCACCAA GCTTCAGATTAG-3′ |
| T2 | 5′-AGAGTCCACCAA GCTACAGATTAG-3′ |
| T3 | 5′-AGAGTCCACCAA GCTCCAGATTAG-3′ |
| T4 | 5′-AGAGTCCACCAA GCTGCAGATTAG-3′ |
| A | 5′-TCTGAAGCT(FAM)TTGATC-Dabsyl-3′ |
| B | 5′-AAAAGATCAAATTGGTGGACTCTAAAA-3′ |

### General Procedures (Unless Specified) of the Restriction Endonuclease Cleavage Reaction:

Probe A (2 μM), probe B (2 μM), template (0-2 uM), BfuCI (0.067 units/uL) in NEBuffer 4 1× (50 mM potassium acetate, 20 mM Tris-aceate, 10 mM magnesium acetate, 1 mM dithiothreitol, pH 7.9) and Bovine Serum Albumin (BSA, 100 μg/ml) at 30° C. for 5 h.

### Gel-Based Read-Out of the Cleavage Reaction:

The reactions were analyzed by 25% denaturing polyacrylamide gel (8 cm×10 cm×1 cm) electrophoresis containing urea (7 M) with Tris-borate-ethylenediaminetetraacetic acid buffer (pH 8.0). The gel was run under 250 V for 1 hr 15 min. After electrophoresis the gel were stained by SYBR Gold. Images were obtained by an STORM scanner and quantified by ImageQuant software (Molecular Dynamics).

### Fluorescence Read-Out of the Cleavage Reaction:

Fluorescence studies were performed on a Hitachi F-4500 spectrometer. The instrument settings were chosen as follows: λ ex=494 nm (slit 5 nm), λ em=504-700 nm (slit 5 nm).

The following examples describe experiments using various probes or probe-analyte complexes (junction probe (JP) tripartites) as outlined in Tables 2 and 3 below. As shown in Tables 2 and 3, a probe analyte complex comprises an analyte (template), a first nucleic acid probe strand (Probe A), and a second nucleic acid probe strand (Probe B), the sequences of which are shown in Table 2.

TABLE 2
DNA sequences of Templates (analytes) and Probe strands

SEQ.
ID NO

Template

| | | |
|---|---|---|
| 1. | T1 | 5′AGAGTCCACCAAGCTTCAGATTAG3′ |
| 2. | T2 | 5′AGAGTCCACCAAGCTCTAGATTAG3′ |

Probe A

| | | |
|---|---|---|
| 3. | Al | 5′TCTGAAGCT^{FAM[a]}TTGATC^{Dabsyl[b]}3′ |
| 4. | A2 | 5′TAATCTGAAGCTTTGATC^{FAM}3′ |
| 5. | A3 | 5′CTAGAGCT^{FAM}TTGATC^{Dabsyl}3′ |
| 6. | A4 | 5′TGAAGCT^{FAM}TTGATC^{Dabsyl}3′ |
| 7. | A5 | 5′GAAGCT^{FAM}TTGATC^{Dabsyl}3′ |

Probe B

| | | |
|---|---|---|
| 8. | B1 | 5′AAAAGATCAAATTGGTGGACTCTAAAA3′ |
| 9. | B2 | 5′AAAGATCAAATTGGTGGACTCTAAAA3′ |
| 10. | B3 | 5′AAGATCAAATTGGTGGACTCTAAAA3′ |
| 11. | B4 | 5′AGATCAAATTGGTGGACTCTAAAA3′ |
| 12. | B5 | 5′GATCAAATTGGTGGACTCTAAAA3′ |
| 13. | B6 | 5′AAAAGATCAAATTGGTGGAC^{FAM}3′ |
| 14. | B7 | 5′AAAA(S)^{[c]}GATCAAATTGGTGGACTCTAAAA3′ |
| 15. | B8 | 5′AAAA(dS)^{[d]}GATCAAATTGGTGGACTCTAAAA3′ |
| 16. | B9 | 5′AAAA(Ac)^{[e]}GATCAAATTGGTGGACTCTAAAA3′ |
| 17. | B10 | 5′AAAA(OMe)^{[f]}GATCAAATTGGTGGACTCTAAAA3′ |
| 18. | B11 | 5′GGGG(S)GATCAAATTGGTGGACTCTAAAA3′ |
| 19. | B12 | 5′CCCC(S)GATCAAATTGGTGGACTCTAAAA3′ |
| 20. | B13 | 5′TTTT(S)GATCAAATTGGTGGACTCTAAAA3′ |
| 21. | B14 | 5′AGTAACGGAAAACCGTTACTAAAA(S)GATCAAATTGGTGGACTCT3′ |
| 22. | B15 | 5′AGTAACGGAAAACCGTTACTAA(S)GATCAAATTGGTGGACTCT3′ |
| 23. | B16 | 5′TGTAACGGAAAACCGTTACA(S)GATCAAATTGGTGGACTCT3′ |
| 24. | B17 | 5′AGTAACGGAAAACCGTTACTAAAA(S)GATCAATTTGGTGGACTCT3′ |
| 25. | B18 | 5′AGTAACGGAAAACCGTTACTAAAA(S)GATCATATTGGTGGACTCT3′ |
| 26. | B19 | 5′TGTAACGGAAAACCGTTACA(S)GATCAATTTGGTGGACTCT3′ |
| 27. | B20 | 5′TGTAACGGAAAACCGTTACA(S)GATCATATTGGTGGACTCT3′ |
| 28. | B21 | 5′TGTAACGGAAAACCGTTACA(S)GATCATTTTGGTGGACTCT3′ |
| 29. | B22 | 5′TG(S)GATCGGAAAACC(S)GATCCA(S)GATCATATTGGTGGACTCT3′ |
| 30. | B23 | 5′TG(S)GATCGGCA(S)GATCGCAAAAGC(S)TGCC(S)GATCCA(S)GA |
| | | TCATATTGGTGGACTCT3′ |
| 31. | B24 | 5′CA(S)GATCGCAAAAGC(S)GATCTGTG(S)GATCGGAAAACC(S)GA |
| | | TCCA(S)GATCATATTGGTGGACTCT3′ |
| 32. | B25 | 5′CT(S)GATCCGAAAACG(S)GATCAGCA(S)GATCGCAAAAGC(S)G |
| | | ATCTGTG(S)GATCGGAAAACC(S)GATCCA(S)GATCATATTGGTGG |
| | | ACTCT3′ |
| 33. | B26 | 5′TG(S)GATCGGCT(S)GATCCGAAAACG(S)GATCAGCA(S)GATCGC |
| | | AAAAGC(S)GATCTGGA(S)GATCGGAAAACC(S)GATCTCCC(S)GAT |
| | | CCA(S)GATCATATTGGTGGACTCT3′ |
| 34. | | 5′AGAGTCCACCAAGCTCTAGATTAG3′ |
| 35. | | 5′TG(S)GATCGGAAAACC(S)GATCCA(S)GATCATATTGGTGGACTCT3′ |
| 36. | | 5′CTAGAGCT^{FAM}TTGATC^{Dabsyl}3′ |
| 37. | | 5′AGAGTCCACCAAGCTCTAGATTAG3′ |
| 38. | | 5′AAAAGATCAAATTGGTGGACTCTAAAA3′ |
| 39. | | 5′CTAGAGCT^{FAM}TTGATC^{Dabsyl}3′ |
| 40. | | 5′^{FAM}TCAAATCTGAAGCTTTGATC^{Dabsyl}3′ |
| 41. | | 5′AGAGTCCACCAAGCTTCAGATTAG3′ |
| 42. | | 5′^{FAM}TCAAATCTGAAGCTTTGATC^{Dabsyl}3′ |
| 43. | | 5′AGAGTCCACCAAGCTTCAGATTAG3′ |
| 44. | | 5′TG(S)GATCGGAAAACC(S)GATCCA(S)GATCATATTGGTGGACTCT3′ |
| 45. | | 5′TCTGAAGCT^{FAM}TTGATC^{Dabsyl}3′ |
| 46. | | 5′AGAGTCCACCAAGCTTCAGATTAG3′ |
| 47. | | 5′GATCAAATTGGTGGACTCTAAAA3′ |
| 48. | | 5′CTAATCTGAAGCTTTGAUCTTTT3′ |
| 49. | | 5′AGAGTCCACCAAGCTTCAGATTAG3′ |
| 50. | | 5′GATCAAATTGGTGGACTCTAAAA3′ |
| 51. | | 5′CTAATCTGAAGCTTTGAUCTTTT3′ |
| 52. | | 5′AGAGTCCACCAAGCTTCAGATTAG3′ |
| 53. | | 5′GATCAAATTGGTGGACTCTAAAA3′ |
| 54. | | 5′CTAATCTGAAGCTTTGAUCTTTT3′ |
| 55. | | 5′AGAGTCCACCAAGCTTCAGATTAG3′ |
| 56. | | 5′GATCAAATTGGTGGACTCTAAAA3′ |
| 57. | | 5′CTAATCTGAAGCTTTGATCTTTT3′ |
| 58. | | 5′GGAGAGUCCACCAAGCUUCAGAUUAG3′ |
| 59. | | 5′GATCAAATTGGTGGACTCTAAAA3′ |
| 60. | | 5′CTAATCTGAAGCT^{FAM}TTGATC^{Dabsy}3′ |

^{[a]}T^{FAM} = Fluorescein-conjugated dT.
^{[b]Dabsyl} = Dabsyl group conjugated at 3′ end.
^{[c]}(S) = phosphorothioate linkage.
^{[d]}(dS) = phosphorodithioate linkage.
^{[e]}(Ac) = phosphoroacetate linkage.
^{[f]}(OMe) = methyl phosphate linkage.

TABLE 3
Components of junction probes (probe-analyte complexes)

| | Template | Probe A | Probe B |
|---|---|---|---|
| JPIa | T1 | A1 | B1 |
| JPIb | T1 | A1 | B2 |
| JPIc | T1 | A1 | B3 |
| JPId | T1 | A1 | B4 |
| JPIe | T1 | A1 | B5 |
| JP1 | T1 | A2 | B6 |
| JP2 | T1 | A1 | B1 |
| JP3 | T1 | A1 | B7 |
| JP4 | T1 | A1 | B8 |
| JP5 | T1 | A1 | B9 |
| JP6 | T1 | A1 | B10 |
| JP7 | T1 | A1 | B11 |
| JP8 | T1 | A1 | B12 |
| JP9 | T1 | A1 | B13 |
| JP10 | T1 | A1 | B14 |
| JP11 | T1 | A1 | B15 |
| JP12 | T1 | A1 | B16 |
| JP13 | T1 | A1 | B17 |
| JP14 | T1 | A1 | B18 |
| JP15 | T1 | A1 | B19 |
| JP16 | T1 | A1 | B20 |
| JP17 | T1 | A1 | B21 |
| JP18 | T1 | A1 | B22 |
| JP19 | T1 | A1 | B23 |
| JP20 | T1 | A1 | B24 |
| JP21 | T1 | A1 | B25 |
| JP22 | T1 | A1 | B26 |
| JP23 | T2 | A3 | B22 |
| JP24 | T1 | A4 | B22 |
| JP25 | T1 | A5 | B22 |

### Example 2

### Cleavage Rate of Junction Probes with Different Modifications at Site B

The probe illustrated in FIG. 6A comprises an analyte (Template), a first nucleic acid probe strand (Probe A), and a second nucleic acid probe strand (Probe B) which has a four base overhang. Together probe A and probe B form a duplex region with the recognition and cleavage site for the REN BfuCI: cleavage site A on probe A and cleavage site B on probe B. The overhang is required for cleavage at the duplex by the REN. Therefore, if cleavage occurs only at site B, removing the overhang, no further cleavage of the probe at site A will occur. If the probe-analyte complex without the overhang is stable, the probes will not dissociate from the analyte, or will do so slowly, stopping or slowing the reaction. Therefore, cleavage site B on probe strand B was modified with nuclease resistant moieties such that only site A was cleaved. FIG. 6B shows the results of various junction probes (JP2-JP6) where N=A (adenosine). JP2-JP6 were modified as follows: JP2: X═O, Y═O⁻; JP3: X═O, Y═S⁻; JP4: X═S, Y═S⁻; JP5: X═O, Y═CH₂COO⁻; and JP6: X═O, Y═OCH₃. The modification of site B of the probe with phosphoromonothioate or phosphorodithioate led to seven and three fold enhancements of the fluorescent intensity, respectively, over the original probe structure wherein site B contained the native phosphate linkage (FIG. 6B). Not all of the nuclease-resistant modifications at site B gave rate enhancement in the restriction endonuclease cleavage reaction. Although the replacement of the exocyclic oxygen in the phosphate linkage at site B with either a methoxy or acetate group completely prevented site B cleavage by BfuCI, the rate of probe-analyte complex cleavage by BfuCI did not differ from that of the native phosphate moiety (FIG. 6B). Therefore, in a preferred embodiment, the cleavage site for the REN is modified such that only one strand of the probe is cleaved.

Without being bound to any one particular theory, it is believed that the results were observed because non-steric mimics of the exocyclic oxygen of the phosphate moiety inhibit either endonuclease/DNA complex formation or the DNA cleavage step. In line with this, phosphoromonothoiate (JP 3) which is a better isostere of phosphate than phosphorodithioate (JP 4) gave a higher rate of cleavage than phosphorodithioate.

FIG. 6C illustrates the results of cleavage reactions for probes (JP3, JP7, JP8, and JP9) with overhangs according to FIG. 6A as follows: JP3: N=A; JP7: N=G; JP8: N═C; and JP9: N=T. For each probe, X═O and Y═S⁻. Probe-analyte complexes with four adenine overhang (JP3) at the 5′-end of probe B was cleaved greater than four times faster than those containing four thymines, cytosines or guanines (JP9, JP8, JP7, respectively; see FIG. 6C).

### Example 3

### Effect of the Duplex Overhang Tail

This Example describes how other architectures, such as duplex overhangs, would influence the restriction endonuclease (REase) cleavage rate of probe-analyte complexes. JP10-14 and JP15-17 (see Tables 2 and 3) were designed to investigate if the presence of a duplex overhang at the 5′-end of probe B would be beneficial to junction probe catalysis. In JP10, 13 and 14, the duplex overhang does not lie immediately after the REase recognition site but is rather appended to a four-nucleotide single-stranded overhang. In contrast, the 5′-duplex overhangs in JP12, 15-17 lie immediately next to the REase recognition site. It must also be noted that whereas JP10-12 contained no mismatched base pairs near the endonuclease cleavage site, for the design of JP 13-17, mismatch base pair sites were introduced at a region that was close to the junction structure. The presence of a duplex overhang at the 5′-end of probe B in the probe-analyte complex enhanced the rate of probe-analyte complex cleavage by the REase, BfuCI. For example, the average rate of cleavage of JP12 (which contains a duplex overhang immediately proceeding the REase duplex recognition site) in the first 5 min was 39 nM/min whereas that of JP3 (which contains a single strand overhang immediately proceeding the REase duplex recognition site) was 0.9 nM/min. Also, the cleavage reaction of JP12 was almost complete after 25 min whereas that of JP3 was not complete after 4 h (240 min, see FIG. 6C). However, the signal-to-noise ratio (S/N; i.e. fluorescent signal generated in the presence of a matching template divided by that generated in the absence of a template) of JP12 was 1.3 after 25 min (when the cleavage reaction is complete for JP12) whereas that of JP3 was 22. Therefore, although the introduction of a duplex overhang next to the REase recognition site significantly accelerated the cleavage reaction, the low S/N prompted us to investigate strategies that would lower the signal-to-noise ratio.

### Example 4

### Effect of the Mismatched Probe B

In order to retain the beneficial effect of placing a duplex overhang next to the cleavage site of BfuCI but increase the S/N, the effect of reducing the base-pair complementarity between probes A and B was studied. One or two, but not three, mismatches were introduced in the duplex region near the endonuclease cleavage site in JP13-17. A maximum of two mismatch base pairs were allowed in the duplex region because the introduction of a mismatch site immediately next to the cleavage site completely abolished the cleavage reaction. The introduction of either a single or double mismatched base-pairs in the duplex region between the probe strands A and B resulted in dramatic changes in S/N. For example JP13/14 and JP10 have similar structures, except that both JP13 and 14 have single mismatch base-pairs in the duplex whereas JP10 does not contain any single mismatch base-pairs, but the S/N of JP13 and 14 after 25 min were 13.5 and 9 respectively whereas that for JP10 was 4.7. Similarly, S/N for JP16 was 10 whereas that for JP12 was significantly lower (1.3), although the two probe designs were similar, except for the presence of a single mismatched base-pair in the duplex region of JP16.

### Example 5

### Effect of Different Duplex Overhang Architectures

Probe-analyte structures JP18-22 were designed to contain an additional recognition site in the duplex overhang at the 5′-end of probe B to investigate if the presence of an additional cognate binding site would allosterically activate the cleavage of probe-analyte structures by BfuCI. As probe B is not cleaved during catalysis, it would be advantageous if probe B remains affixed to the target analyte during the catalytic process. Therefore, it is desirable that the second REase cognate recognition site that is placed on the 5′-duplex overhang of probe B contains nuclease resistance sites so that the duplex moiety remains intact during catalysis. Consequently, in JP18-22, the second recognition site on the duplex overhang was modified with phosphoromonothioate at the two cleavage sites. The initial rate of cleavage of JP18 was 82 nM/min, whereas the initial rate of REase cleavage of JP16 was 43 nM/min; indicating that BfuCI might belong to the growing class of type II REases that are allosterically activated by a second recognition site. The addition of a third recognition site in the duplex overhang region of probe B (see JP19) did not lead to any further enhancement of cleavage rate. The architecture of the 5′-overhang is also important for JP cleavage. Overhangs that contain additional recognition sites but that differ in structure from the canonical duplex overhang in JP18 led to a dramatic decrease in cleavage rate; indicating that steric encumbrance is not tolerated by the enzyme (JP 20-22).

### Example 6

### Effect of the Length of Template Hybridization Region of Probe A

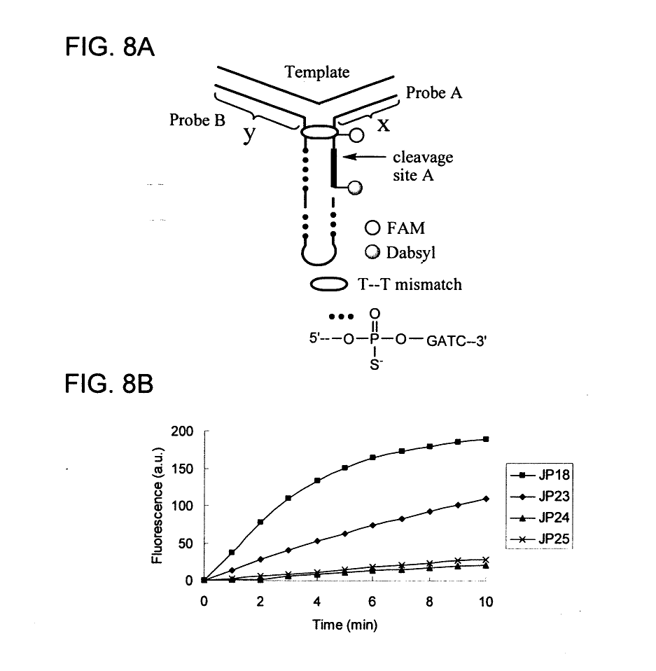

The effect of probe strand A-template base-pairing number (the number of base pairs between probe strand A and the template) on the junction probe cleavage rate was investigated. Probe-analyte structures JP18 and JP23-25 were designed to have various lengths of hybridization between the template (analyte) and probe strand A (probe A) of the probe (FIG. 8A). The junction probes have the following lengths (number of nucleotides) of hybridization (X): JP18: X=8; JP23: X=7; JP24: X=6; JP25: X=5. Segment Y of probe B (FIG. 8A) was 12 nucleotides for each probe. The effect of the length of template hybridization to probe A was determined by measuring the rate of cleavage (FIG. 8B). When template/probe strand A base pairing was equal to or greater than 7, practical catalytic efficiency was obtained. The results indicate that the on-rate of probe strand A/template/probe strand B complex is important for junction probe catalysis. Without being bound to any one particular theory, it is believed that the results were observed because the longer region of hybridization results in a higher melting temperature for JP18, which can result in a more stable probe-analyte complex, which can in turn result in a higher rate of cleavage of the probe-analyte complex.

### Example 7

### Comparison of Junction Probes to Molecular Beacons

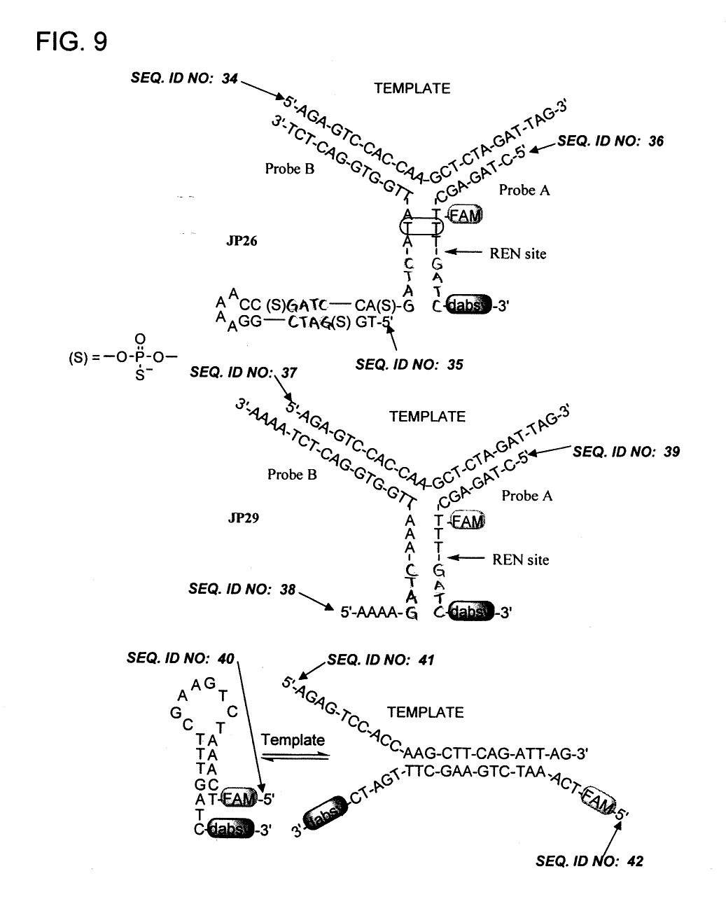

Junction probes were compared to molecular beacons in their abilities to detect analytes by measuring the fluorescence generated in the presence of a template (analyte). (FIG. 9). Templates were detected either using junction probe technology or molecular beacon ((FLUORESCEIN)TCAAATCTGAAGCTTTGATC (DABSYL)-3′) technology, which provides a maximum of one signal per template. The fluorescence signal that was generated from the junction probe platform was 373 times more intense than that of molecular beacon technology for JP26 and 43 times more intense than that of molecular beacon technology for JP29. The results demonstrate that the signal generated using junction probe technology was significantly greater than the signal generated using molecular beacon technology and that signal amplification was obtained at isothermal conditions using junction probe technology.

The following Example describes the material and methods used in the experiments described in Examples 2-7.

### Example 8

General procedures: DNA Template, probe A and probe B in 60 μL of NE buffer 4 1× (50 mM potassium acetate, 20 mM Tris-aceate, 10 mM magnesium acetate, 1 mM dithiothreitol, pH 7.9) with Bovine Serum Albumin (BSA, 100 μg/ml) were mixed at 30° C., and the fluorescence at time=0 min (referred to as t₀) was recorded. BfuCI (0.5 μL 0.067 units/μL) was then added to the solution and fluorescence was taken every 1 min. Fluorescence studies were performed on a Cary Eclipse fluorescence spectrophotometer.

Gel-based analysis of the cleavage reaction: The reaction conditions were the same as described in general procedures. The 25% denaturing polyacrylamide gel (8 cm×10 cm×1 cm) containing urea (7 M) was run under 250 V for 55 min in Tris borate-ethyleniamine tetraacetic acid buffer (pH 8.0). After electrophoresis the gel were stained with SYBR Gold stain. Images were obtained with STORM scanner (Molecular Dynamics).

### Example 9

### G-quadruplex DNAzyme-Junction Probe Technology

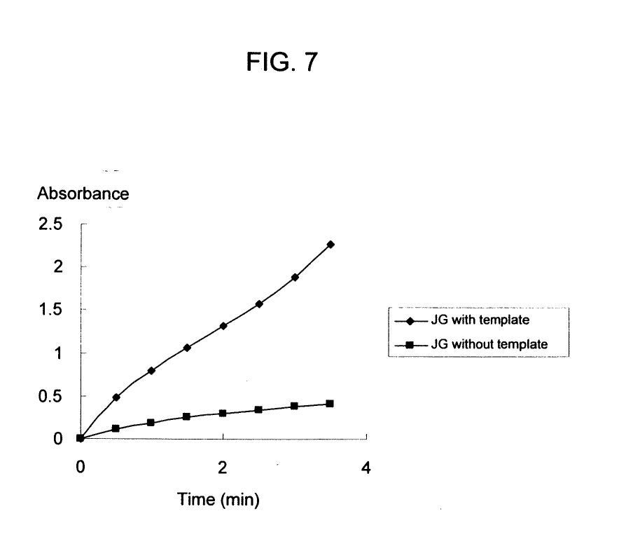

The probe illustrated in FIG. 2 was used. The probe comprised two nucleic acid strands, Probe A and Probe B. Probe A was designed to comprise a locked G-quadruplex DNAzyme that is inactive as a peroxidase-mimicking DNAzyme. In the absence of a template, probe A and probe B did not associate to any appreciable extent. However, when a template analyte was present a ternary structure was formed. The ternary structure reconstituted a restriction endonuclease cleavage site and the subsequence cleavage by Bfcul led to the destruction of the stem-loop probe A into two halves that fell off the ternary structure. Once probe A was cleaved into two halves, a G-quadruplex DNAzyme readily formed. This G-quadruplex DNAzyme was detected colorimetrically via its peroxidation of the chromogenic substrate ABTS. As shown in the absorbance spectra (FIG. 7), this technique discriminated the presence of a template (diamonds) from the absence of a template (squares) when absorbance was measured over time. In addition, the detection probes did not require fluorescent labeling.

### Example 10

### Cleavage of Junction Probes Using Different Endonucleases

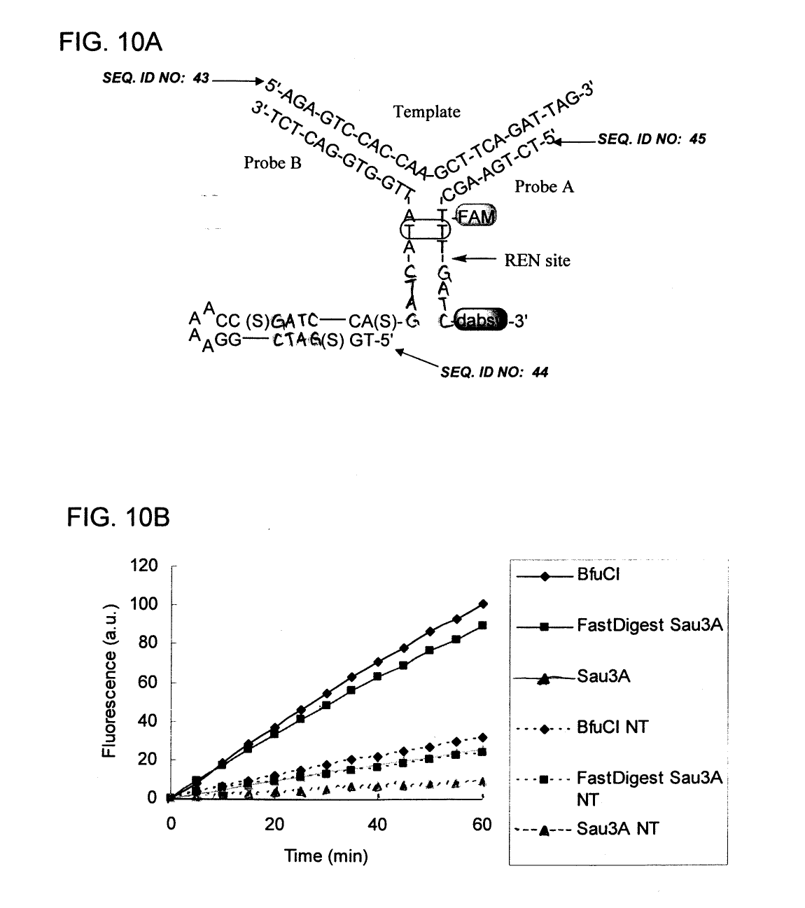

Junction probes were designed and tested for their ability to be cleaved by various restriction endonucleases (BfuCI, FastDigest Sau3A and Sau3A; FIG. 10A). The rate of cleavage was measured by detecting fluorescent intensities over time as described, for example, in Example 8. All enzymes were used at their optimized concentration, respectively (BfuCI 0.165 U/μL, FastDigest Sau3A 0.0417 U/μL, Sau3A 0.333 U/μL). Fluorescence data was normalized (λ ex=494 nm, λ em=522 nm, Ex slit=Em slit=10 nm, PMT=600 V). The reaction conditions were as follows: 200 nM probes A and B respectively, 1 nM template, 30° C. NT indicates no template was added. All three enzymes tested cleaved at a higher rate in the presence of template as compared to when no template was added to the reaction (FIG. 10B). The results indicate that junction probes can be designed to work with various restriction endonucleases.

### Example 11

### Cleavage of Junction Probes Using Different Endonucleases

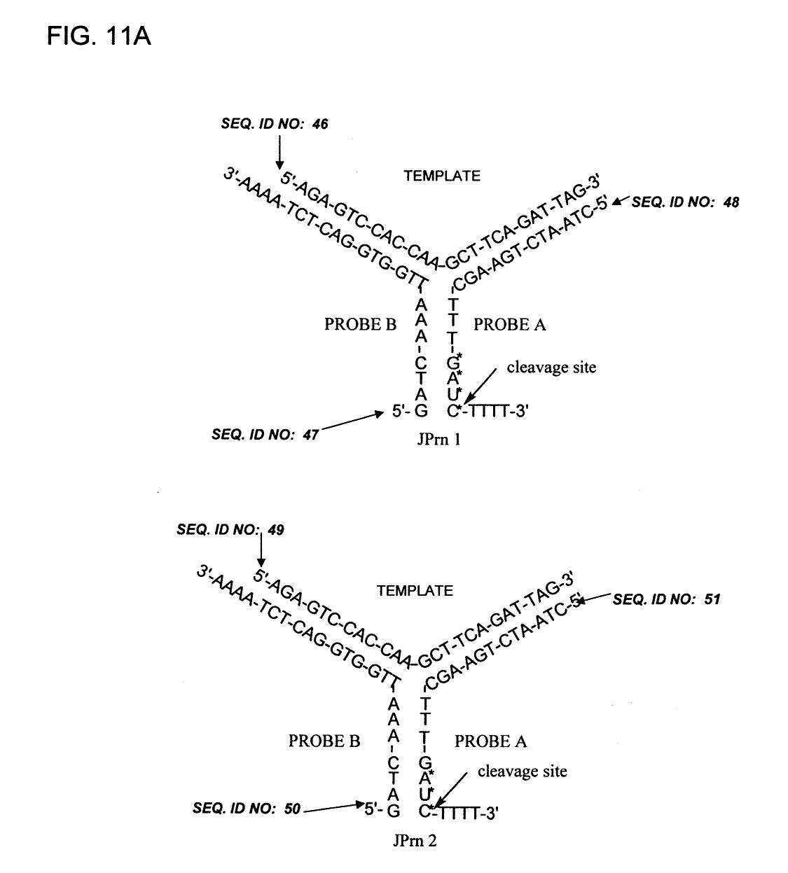

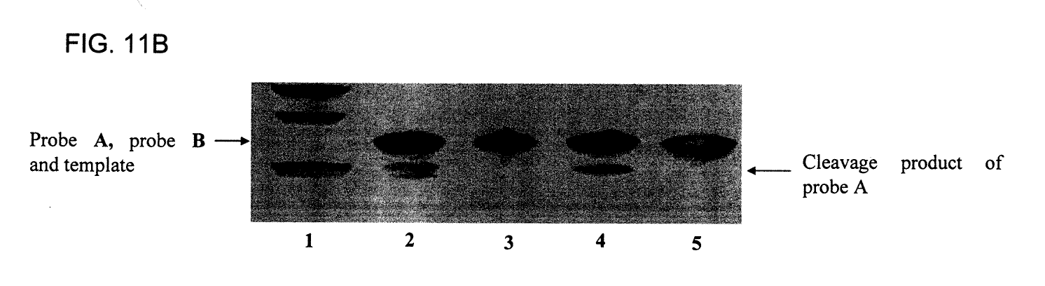

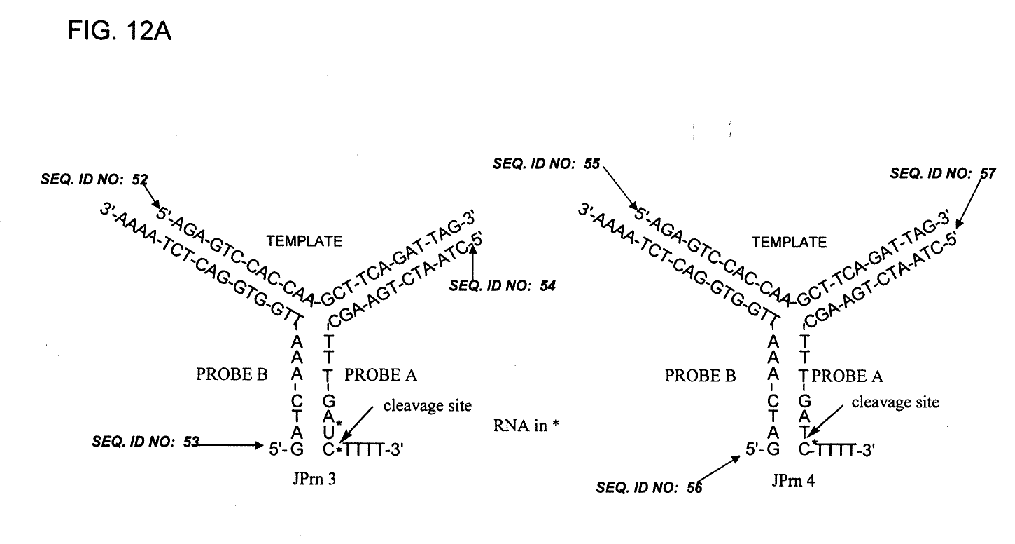

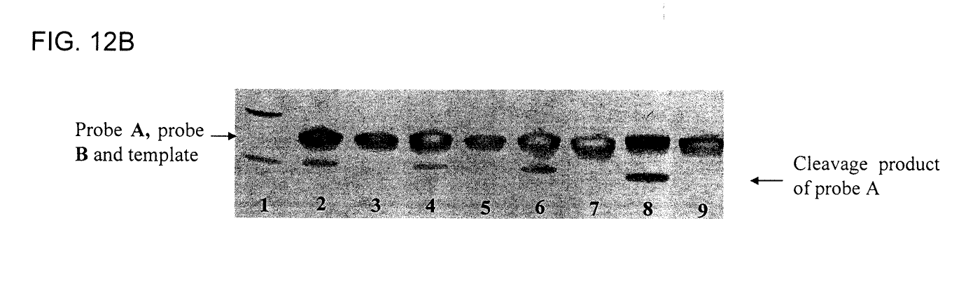

Junction probes (JPrn 1-4) were designed and tested for their ability to be cleaved by RNase H or RNase HII (FIGS. 11A and 12A, respectively). Nucleotides illustrated with an asterisk (*) in FIGS. 11A and 12A represent RNA. The reaction conditions were as follows: 2 μM probes A and B, respectively; 2 μM template; 0.067 U/μL of RNase H or RNase HII; 30° C. for 12 h. Cleavage was measured by detecting cleavage products on a PAGE gel (FIGS. 11B and 12B) in the presence or absence of template as described, for example, in Example 8. RNase H cleaved probe A of JPrn 1 (lane 2, FIG. 11B) and JPrn 2 (lane 4, FIG. 11B) when probe B and template was present, but did not cleave probe A of JPrn 1 (lane 3, FIG. 11B) and JPrn 2 (lane 5, FIG. 11B) when template was not present. A 20, 30, and 40mer marker was run in lane 1 (FIG. 11B). RNase HII cleaved probe A of JPrn 1 (lane 2, FIG. 12B), JPrn 2 (lane 4, FIG. 12B), JPrn 3 (lane 6, FIG. 12B), and JPrn 4 (lane 8, FIG. 12B) when probe B and template was present, but did not cleave probe A of JPrn 1 (lane 3, FIG. 12B), JPrn 2 (lane 5, FIG. 12B), JPrn 3 (lane 7, FIG. 12B), JPrn 4 (lane 9, FIG. 12B) when template was not present. A 20 and 30mer marker was run in lane 1 (FIG. 12B). All junction probes tested produced more cleavage products in the presence of RNase H or RNase HII and template as compared to when no template was added to the reaction (FIGS. 11B and 12B). The results indicate that junction probes can be designed to work with various endonucleases.

### Example 12

### Detecting mircoRNA with Junction Probe Technology

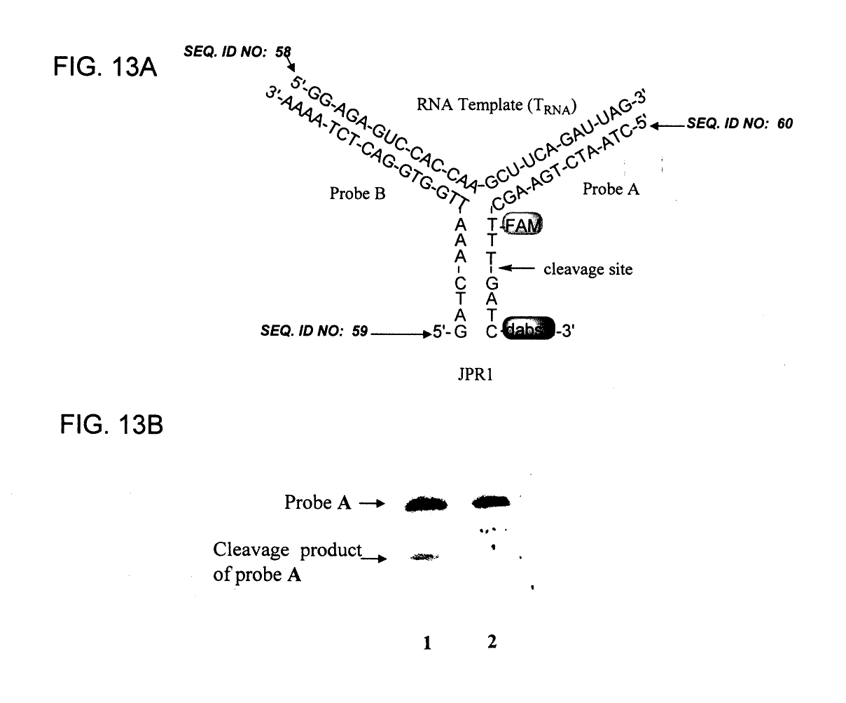

Junction probes (JPR 1) were designed and tested for their ability to detect microRNA templates (FIG. 13A). The microRNA substrate was prepared using RNA synthesis methods known in the art. Briefly, a DNA template containing a T7 promoter sequence was transcribed with a T7 RNA polymerase. The DNA template was then digested with DNase and the RNA isolated via ethanol precipitation. Cleavage was measured by detecting cleavage products on a PAGE gel (FIG. 13B) in the presence or absence of template as described, for example, in Example 8. Cleavage products of probe A of JPR 1 were detected in the presence of microRNA template (FIG. 13B, lane 2) as compared to when no RNA template was added to the reaction (FIG. 13B, lane 2). The results indicate that junction probes can be designed to detect various nucleic acid templates.

While the present teachings have been described in terms of these exemplary embodiments, the skilled artisan will readily understand that numerous variations and modifications of these exemplary embodiments are possible without undue experimentation. All such variations and modifications are within the scope of the current teachings.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein.

Unless otherwise indicated, the singular use of various words, including the term “an” or “an” denotes both the option of a single or more than one. In addition, the use of the term “and/or” denotes various embodiments that include: both options, either option in the alternative, or the combination of either option in the alternative and both options. When describing various combinations, kits, probes, methods, etc., it will be understood that unless otherwise stated, the combinations are described as comprising, consisting of, and consisting essentially of. This does not apply to the claims or to situations in the specification where the term “consisting of” is used.

### INCORPORATION BY REFERENCE

All references cited herein, including patents, patent applications, papers, text books, and the like, and the references cited therein, to the extent that they are not already, are hereby incorporated by reference for the subject matter referenced herein, and in their entirety. In the event that one or more of the incorporated literature and similar materials differs from or contradicts this application, including but not limited to defined terms, term usage, described techniques, or the like, this application controls.

### EQUIVALENTS

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A probe for detecting a nucleic acid analyte in a sample, comprising:
(a.) a first nucleic acid probe strand comprising:
a first cleavage region comprising a first cleavage site sequence; and
a first analyte recognition region that is substantially complementary to a first portion of said nucleic acid analyte; and
(b.) a second nucleic acid probe strand comprising:
a second cleavage region that is substantially complementary to said first cleavage region of said first nucleic acid probe strand; and
a second analyte recognition region that is substantially complementary to a second portion of said nucleic acid analyte;
wherein said probe is configured such that in the presence of said nucleic acid analyte, said first nucleic acid probe strand, said second nucleic acid probe strand and said nucleic acid analyte can hybridize to each other to form a probe-analyte complex, wherein:
said first analyte recognition region of said first nucleic acid probe strand is hybridized to said first portion of said nucleic acid analyte to form a first duplex region;
said second analyte recognition region of said second nucleic acid probe strand is hybridized to said second portion of said nucleic acid analyte to form a second duplex region; and
said first cleavage region of said first nucleic acid probe strand is hybridized to said second cleavage region of said second nucleic acid probe strand to form a third duplex region, thereby forming a first cleavage site in said first cleavage region of said first nucleic acid probe strand; and
wherein the melting temperature of said probe-analyte complex is greater than the melting temperature of a duplex of said first and second nucleic acid probe strands in the absence of said nucleic acid analyte.

2. The probe of claim 1, wherein said first nucleic acid probe strand further comprises a detectable moiety.

3. The probe of claim 2, wherein said detectable moiety comprises a fluorophore.

4. The probe of claim 3, wherein said first nucleic acid probe strand further comprises a quencher.

5. The probe of claim 4, wherein one of said fluorophore and said quencher are located upstream of said first cleavage site and the other is located downstream of said first cleavage site.

6. The probe of claim 2, wherein said detectable moiety comprises a biotin molecule.

7. The probe of claim 6, wherein said first nucleic acid probe strand further comprises a solid support.

8. The probe of claim 1, wherein said first cleavage site is located in said third duplex region.

9. The probe of claim 1, wherein said first cleavage site is located about 1 to about 6 nucleotides from the 5′ end or the 3′ end of said first cleavage region of said first nucleic acid probe strand.

10. The probe of claim 1, wherein said first cleavage site is located about 3 nucleotides from said 5′ end of said first cleavage region of said first nucleic acid probe strand.

11-36. (canceled)

37. A method for detecting a nucleic acid analyte in a sample, comprising:
contacting said sample with a probe, wherein said probe comprises:
(a.) a first nucleic acid probe strand comprising:
a first cleavage region comprising a first cleavage site sequence; and
a first analyte recognition region that is substantially complementary to a first portion of said nucleic acid analyte; and
(b.) a second nucleic acid probe strand comprising:
a second cleavage region that is substantially complementary to said first cleavage region of said first nucleic acid probe strand; and
a second analyte recognition region that is substantially complementary to a second portion of said nucleic acid analyte;
forming a probe-analyte complex in the presence of said nucleic acid analyte wherein:
said first analyte recognition region of said first nucleic acid probe strand is hybridized to said first portion of said nucleic acid analyte to form a first duplex region;
said second analyte recognition region of said second nucleic acid probe strand is hybridized to said second portion of said nucleic acid analyte to form a second duplex region; and
said first cleavage region of said first nucleic acid probe strand is hybridized to said second cleavage region of said second nucleic acid probe strand to form a third duplex region, thereby forming a first cleavage site in said first cleavage region of said first nucleic acid probe strand; and
wherein the melting temperature of said probe-analyte complex is greater than the melting temperature of a duplex of said first and second nucleic acid probe strands in the absence of said nucleic acid analyte;
providing an enzyme that cleaves said first nucleic acid probe strand at said first cleavage site, wherein said first nucleic acid probe strand is cleaved into a first fragment and a second fragment; and
detecting said cleavage of said first nucleic acid probe strand, thereby detecting the presence of said nucleic acid analyte.

38. The method of claim 37, wherein said first nucleic acid probe strand comprises a detectable moiety.

39. The method of claim 38, wherein said first fragment of said first nucleic acid probe strand comprises said detectable moiety.

40. The method of claim 38, wherein said detectable moiety comprises a fluorophore and said second fragment of said first nucleic acid probe strand comprises a quencher.

41. The method of claim 38, wherein said detectable moiety comprises a fluorophore and said second fragment of said first nucleic acid probe strand comprises a fluorophore.

42. The method of claim 38, wherein said detectable moiety comprises a biotin molecule.

43. The method of claim 37, wherein said enzyme comprises a endonuclease.

44. The method of claim 43, wherein said endonuclease is a restriction endonuclease.

45-47. (canceled)

48. A method for detecting a nucleic acid analyte in a sample, comprising:
contacting said sample with a probe, wherein said probe comprises:
(a.) a first nucleic acid probe strand comprising:
a first fragment of an enzyme;
a first probe recognition region; and
a first analyte recognition region that is substantially complementary to a first portion of said nucleic acid analyte;
(b.) a second nucleic acid probe strand comprising:
a second fragment of said enzyme;
a second probe recognition region that is substantially complementary to said first probe recognition region of said first nucleic acid probe strand; and
a second analyte recognition region that is substantially complementary to a second portion of said nucleic acid analyte;
wherein said probe is configured such that in the presence of said nucleic acid analyte, said first nucleic acid probe strand, said second nucleic acid probe strand and said nucleic acid analyte can hybridize to each other to form a probe-analyte complex, wherein:
said first analyte recognition region of said first nucleic acid probe strand is hybridized to said first portion of said nucleic acid analyte to form a first duplex region;
said second analyte recognition region of said second nucleic acid probe strand is hybridized to said second portion of said nucleic acid analyte to form a second duplex region;
said first probe recognition region of said first nucleic acid probe strand is hybridized to said second probe recognition region of said second nucleic acid probe strand to form a third duplex region, thereby reconstituting said enzyme; and
wherein the melting temperature of said probe-analyte complex is greater than the melting temperature of a duplex of said first and second nucleic acid probe strands in the absence of said nucleic acid analyte; and
measuring said enzyme activity, thereby detecting the presence of said nucleic acid analyte.

49. The method of claim 48, wherein said enzyme comprises G-quadruplex DNAzyme.
